(19) United States
(12) Patent Application Publication
(10) Pub. No.: US 2025/0345295 A1
(43) Pub. Date: Nov. 13, 2025

(54) LMK235 COMPOSITIONS AND METHODS

(71) Applicant: UNM Rainforest Innovations, Albuquerque, NM (US)

(72) Inventors: Karin Westlund HIGH, Albuquerque, NM (US); Sascha ALLES, Albuquerque, NM (US); SiFong E. HUI, Albuquerque, NM (US)

(21) Appl. No.: 19/201,488

(22) Filed: May 7, 2025

Related U.S. Application Data
(60) Provisional application No. 63/645,570, filed on May 10, 2024.

(51) Int. Cl. A61K 31/166; A61P 25/02

(52) U.S. Cl. CPC A61K 31/166; A61P 25/02

(57) ABSTRACT

A method of treating neuropathic pain in a subject generally includes administering to the subject an amount of LMK235 effective to alleviate neuropathic pain experienced by the subject. In one or more embodiments, the neuropathic pain is chronic. A method of suppressing HDAC5 expression in a cell generally includes contacting the cell with an amount of LMK235 effective to suppress HDAC5 expression. A method of treating a subject having or at risk of having a condition characterized at least in part by HDAC5 expression generally includes administering to the subject an amount of LMK235 effective to suppress expression of HDAC5 is the subject's tissues.

### CROSS-REFERENCE TO RELATED APPLICATION

This application claims the benefit of U.S. Provisional Patent Application No. 63/645,570, filed May 10, 2024, which is incorporated herein by reference in its entirety.

### GOVERNMENT FUNDING

This invention was made with government support under W81XWH-20-1-0930 awarded by the Medical Research and Development Command, and NS123958 and DE028096 awarded by the National Institutes of Health. The government has certain rights in the invention.

### SUMMARY

This disclosure describes, in one aspect, a method of treating neuropathic pain in a subject. Generally, the method includes administering to the subject an amount of LMK235 effective to alleviate neuropathic pain experienced by the subject.

In one or more embodiments, the neuropathic pain is chronic.

In one or more embodiments, the effective amount of LMK235 is an amount effective to reduce mechanical sensitivity in the subject compared to a vehicle-treated control.

In one or more embodiments, the effective amount of LMK235 is an amount effective to reduce cold sensitivity in the subject compared to a vehicle-treated control.

In one or more embodiments, the effective amount of LMK235 is an amount effective to reduce anxiety-related behaviors associated with experiencing neuropathic pain compared to a vehicle-treated control.

In one or more embodiments, the effective amount of LMK235 is an amount effective to reduce excitability of isolated trigeminal ganglia neurons from an injured nerve injured compared to a vehicle-treated control.

In one or more embodiments, the effective amount of LMK235 is an amount effective to reverse one or more epigenetic change that resulted from injury to the injured nerve.

In one or more embodiments, the effective amount of LMK235 is an amount to reduce sag ratio in trigeminal neurons compared to injured trigeminal neurons untreated with LMK235.

In another aspect, this disclosure describes a method of suppressing histone deacetylase 5 (HDAC5) expression in a cell. Generally, the method includes contacting the cell with an amount of LMK235 effective to suppress expression of HDAC5.

In another aspect, this disclosure describes a method of suppressing histone deacetylase 5 (HDAC5) expression in a subject. Generally, the method includes administering LMK235 to the subject in an amount of LMK235 effective to suppress expression of HDAC5 in one or more tissues of the subject.

In one or more embodiments, the subject has, or is at risk of having, a condition caused at least in part by overexpression of HDAC5.

In one or more of these embodiments, the condition is diabetic neuropathy, myelofibrosis, periodontitis, osteoporosis, osteopetrosis, bacterial-induced osteolysis, nerve injury, brain injury, systemic sclerosis, rheumatoid arthritis, diabetic kidney disease, acute kidney injury, polycystic kidney disease, polycystic ovary, or an inflammatory gastrointestinal syndrome.

In one or more of these embodiments, the condition is a cancer.

In one or more of these embodiments, administering LMK235 to the subject improves cardiac dysfunction, pathological ventricular remodeling, or both.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. The HDAC5 inhibitor, LMK235, altered gene transcription allowing the reversal of the cycle driving the chronic trigeminal injury-induced neuropathic pain. (A) Chemical structure of LMK235. (B) Schematic illustration of the mechanism by which LMK235 relieves neuropathic pain.

FIG. 2. LMK235 (5 mg/kg) durably reverses mechanical and cold hypersensitivity in two chronic trigeminal nerve injury models, but no abuse potential was indicated. (A) Two chronic trigeminal nerve injury models. Placement of chromic gut suture at two different sites along the infraorbital branch of the maxillary trigeminal nerve induces the TIC (site #1) and FRICT-ION (site #2) chronic craniofacial pain models. (B) C57B1/6 mice with TIC: Mechanical hypersensitivity was reversed to naïve baseline with HDAC5 inhibitor LMK235 post-treatment in week 3 (daily, 5 mg/kg, s.c.) compared to injured male mice treated with vehicle only. (C) C57B1/6 mice with TIC: Mechanical hypersensitivity was significantly reversed even when LMK235 was given eight weeks after induction of the chronic trigeminal pain model compared to injured male mice treated with vehicle only. ANOVA *p>0.05, ****p<0.001.

FIG. 3. LMK235 (5 mg/kg) reverses mechanical and cold hypersensitivity in the chronic trigeminal nerve injury models, in both male and female mice. BALBc mice with FRICT-ION: LMK235 reverses von Frey hypersensitivity in mice with FRICT-ION treated daily in week 3 (5 mg/kg, s.c), ANOVA ****p<0.001 compared to FRICT-ION+vehicle.

FIG. 4. LMK235 (5 mg/kg) reverses cold hypersensitivity in the chronic trigeminal nerve injury models. (A) Cold probe testing indicates cold hypersensitivity is significantly improved after LMK235 treatment (week 6) ANOVA ***p>0.001 compared to naïve mice, and #p<0.05 compared to FRICT-ION+vehicle. (B) The conditioned place preference (CPP) test indicates LMK235 has no addiction potential ANOVA ****p<0.0001 compared to FRICT-ION+vehicle.

FIG. 5. LMK235 reduces anxiety-like behavior. At eight weeks post FRICT-ION trigeminal nerve injury, LMK235 reduction of anxiety measures was confirmed with the light/dark box test. ANOVA *p<0.05, **p<0.01, compared to naïve mice.

FIG. 6. LMK235 reduces anxiety-like behavior. At eight weeks post FRICT-ION trigeminal nerve injury, LMK235 reduction of anxiety measures was confirmed with zero maze test. ANOVA *p<0.05, **p<0.01, compared to naïve mice.

FIG. 7. In vitro LMK235 reduces excitability of small-diameter trigeminal ganglia from FRICT-ION, but not naïve mice. (A) Electrophysiological properties from naïve mice were not significantly altered by LMK235 treatment. (B) Electrophysiological properties from FRICT-ION mice were altered by LMK235 treatment. RMP and average rheobase were not significantly altered. Distribution of high (>200 pA) vs low (<200 pA) threshold TG neurons was altered in FRICT-ION mice. LMK235-treated TG cultures had a higher proportion of high threshold (>200 pA) neurons compared to controls (p<0.05, Fisher's exact test, n=21-23 neurons per condition). Sag ratio was significantly lower (**p<0.001, Mann-Whitney test) in LMK235-treated neurons compared to controls. n=8-19 neurons per condition. Control=0.1% DMSO for one hour, LMK235=13 μM LMK235 for one hour.

FIG. 8. GO analysis for differentially expressed genes in TG of male FRICT-ION mice compared to naïve controls at week 10.

FIG. 9. GO analysis for differentially expressed genes in TG obtained from male FRICT-ION mice treated with HDAC5 inhibitor, LMK235, compared to naïve controls at week 10.

FIG. 10. GO analysis for differentially expressed genes in TG obtained from male FRICT-ION mice treated with HDAC5 inhibitor, LMK235, compared to FRICT-ION mice treated with vehicle at week 10.

FIG. 11. Heat maps of differential gene expression. The heat maps of pain related genes using categories from the GO analyses for the TG from male LMK235-treated and untreated FRICT-ION mice compared to naïve mice or to each other at week 10, p<0.05 was considered significant. Only genes upregulated two-to-five-fold are included with those in red upregulated and those in blue downregulated. The genes were grouped into six categories for the heat map most relevant to pain which included G protein-related peptides, hormones/neurotransmitters, immunity/inflammatory/cytokines, ion channel/ion binding/transport, neurogenesis/axon regeneration/axogenesis/neuron differentiation/, and transcription/regulation of gene expression/histones. The comparison of LMK235-treated FRICT-ION mice with naïves displayed a notable lack of genes altered. The largest collections of pain-related genes were the transcription/regulation of gene expression/histones grouping and the transcription/regulation of genes grouping. Immunity/inflammatory/cytokine genes were also prominent.

FIG. 12. RT-PCR data showing the FRICT-ION nerve injury induced increase of HDAC5, GNB2, and CCKBR gene expression in week 10.

FIG. 13. HDAC5 RNA and protein levels are increased in LMK235-treated mice. (A) RT-PCR data showing the nerve injury induced increase of HDAC5 gene expression evident in week 10 in male FRICT-ION mice, ANOVA *p<0.05, **p<0.01, ***p<0.001, ****p<0.0001, and p<0.05 compared to LMK235-treated naïve or FRICT-ION+LMK235 mice. (B) HDAC5 protein levels are increased in FRICT-ION mice and reduced in LMK235-treated mice with FRICT-ION. Western blot confirmed HDAC5 protein was increased in the trigeminal ganglia of FRICT-ION mice. TG HDAC5 protein levels were increased in FRICT-ION male (***p<0.001) and female mice (****p<0.0001) compared to naïve controls. LMK235 treatment significantly reduced TG HDAC5 protein levels to naïve levels in females and below naïve levels in males (**p<0.01). LMK235-treated FRICT-ION mice had significantly lower HDAC protein levels than untreated FRICT-ION mice (****p<0.0001 in males, ***p<0.001 in females).

FIG. 14. Heat maps showing differential gene expression in TG at week 10. (A) Naïve versus LMK235-treated FRICT-ION mice. (B) Naïve versus LMK235-untreated FRICT-ION mice. (C) LMK235-treated FRICT-ION mice versus LMK-untreated FRICT-ION mice. Over 200 genes were significantly upregulated and >200 were downregulated in FRICT-ION mice compared to naïves. Fewer genes (120) were upregulated in the FRICT-ION mice treated with LMK235 compared to those untreated, and fewer were downregulated (148). Many fewer genes (84 genes) were significantly upregulated or downregulated (68) in FRICT-ION mice treated with LMK235 compared to the naïve group. p<0.05 was considered significant.

FIG. 15. Volcano plot of differential gene expression in TG from LMK235-untreated FRICT-ION mice compared to naïve mice at week 10.

FIG. 16. Volcano plot of differential gene expression in TG from LMK235-treated FRICT-ION mice compared to LMK235-untreated FRICT-ION mice at week 10.

FIG. 17. Volcano plot of differential gene expression in TG from LMK235-treated FRICT-ION mice compared to naïve mice at week 10.

### DETAILED DESCRIPTION OF ILLUSTRATIVE EMBODIMENTS

This disclosure describes a method of treating neuropathic pain in a subject. The neuropathic path may be chronic. Generally, the method includes administering to the subject an amount of LMK235 effective to alleviate neuropathic pain experienced by the subject. Without wishing to be bound by any particular theory, the methods described herein reduce the nerve activation that causes neuropathic pain, thereby producing durable reduction, including but not limited to reversal, of the chronic pain.

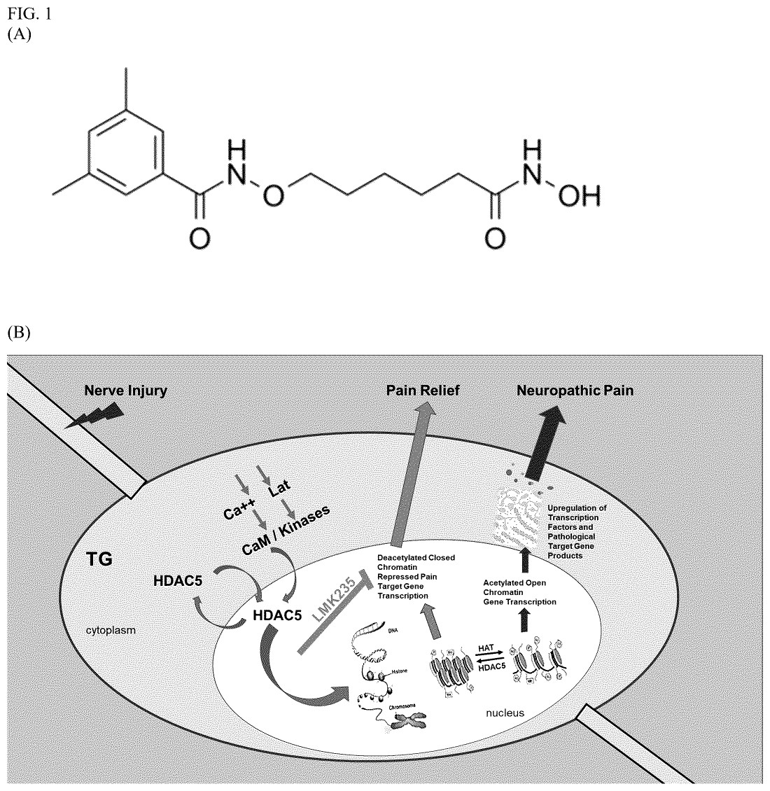

Identifying and resolving the complexities underlying chronic neuropathic pain is a significant challenge. The present disclosure describes methods for treating pain using the Class IIa HDAC4/5 inhibitor, LMK235 (FIG. 1A). The efficacy of LMK235 in treating neuropathic pain was demonstrated over seven weeks using a chronic trigeminal nerve injury FRICT-ION (foramen rotundum inflammatory compression trigeminal infraorbital nerve) model.

Epigenetic regulation affects long-term expression of genes through direct modification (e.g., acetylation, methylation, ubiquitination, and/or phosphorylation) of histones, the alkaline proteins that package DNA. Histone acetylation and deacetylation are mechanisms in transcription, allowing access to specific DNA regions, chromatin remodeling, cell cycle, signal transduction, and control of gene expression. The interplay between the epigenetic modulators histone acetyltransferases (HATs) and histone deacetylases (HDACs) is dynamically balanced to maintain homeostasis. While HATs render chromosomes more accessible for transcription, actions of HDACs lead to compaction of DNA and restraint of its transcription. HDACs are highly expressed in nervous tissue and increased expression is observed in injured dorsal root ganglia (DRG) and spinal cord after noxious formalin or following spinal nerve ligation. While HDACs typically reside either in the nucleus or the cytoplasm, the Class IIa HDACs are unusual in that they actively shuttle between the nucleus and the cytoplasm.

While HDAC2 is reported to be prominent in DRG in the spinal nerve ligation model (SNL) at 3 weeks, the RNAseq and Western blot data provided herein show significant two-fold increased expression of HDAC5 mRNA and protein in week 10 in mice with chronic trigeminal nerve injury-induced neuropathic pain. The significant increase in HDAC5 RNA and protein are not found in mice treated with Class IIa HDACi LMK235. The LMK235 given as a post-treatment (e.g., week 3 or week 8) effectively reduces mechanical and cold hypersensitivity compared to vehicle treatment in both male and female mice in both the trigeminal CCI and FRICT-ION neuropathic pain models. Non-evoked pain-related anxiety behaviors fail to develop in LMK235-treated mice as a result. Study of TG neuron primary cultures from the treated mice indicate LMK235 reduces the excitability of TG neurons from FRICT-ION mice.

Thus, while it is known nerve injury enhances direct modification of histones by HDACs inhibiting gene transcription (FIG. 1), the actions of HDACi LMK235 effectively diminished HDAC5 protein, HDAC5 RNA, and promoted or inhibited many other gene alterations. The net result was a reduction of the TG neuronal responses and reversal of behavioral hypersensitivity that inhibited the development of anxiety behaviors in the chronic trigeminal neuropathic pain model. The findings indicate that LMK235 can effectively alleviate neuropathic pain.

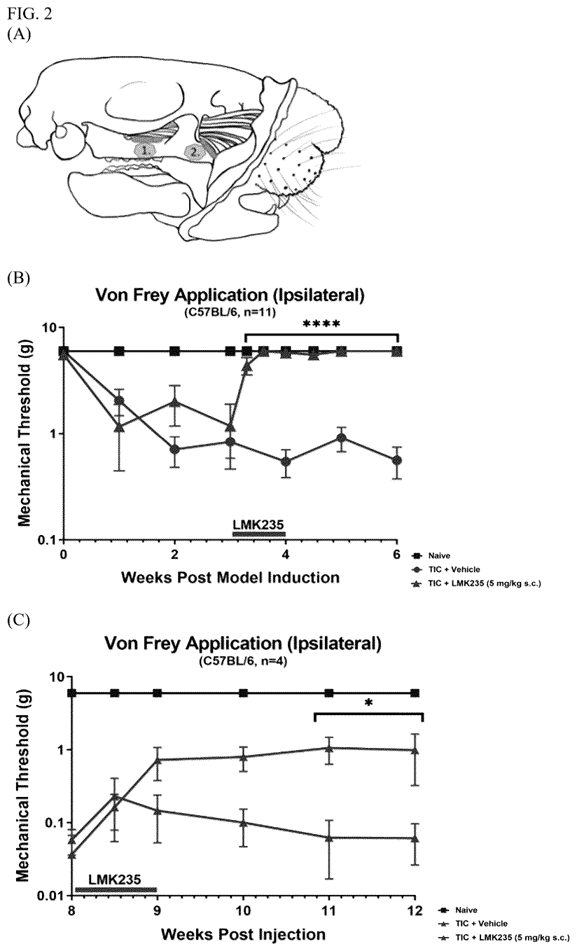

One obstacle to better understanding of pathophysiological mechanisms of chronic neuropathic pain has been infrequent use of experimental animal models that mimic chronic pain symptoms, defined as persisting >12 weeks in patients. To emulate chronic craniofacial neuropathic pain, two mouse models have been developed with mechanical and cold hypersensitivity similar to patient complaints. Since microvascular decompression surgery is reportedly often successful in relieving chronic craniofacial pain, both clinically relevant models mimic the common etiology of microvascular compression rather than nerve ligation. The models display the neuropathic mechanical hypersensitivity and also the cold hypersensitivity experienced by patients with trigeminal neuralgia (FIG. 2). Occasional episodic eye wincing behavior is noted reminiscent of the brief electric shock-like (lancinating) pains experienced by patients with trigeminal neuralgia.

### Induction of the Clinically Relevant Models of Chronic Trigeminal Neuropathic Pain

The mouse models are induced surgically by inserting chromic gut suture into the tight space between the infraorbital nerve (ION) branch of the trigeminal nerve either at the bony infraorbital fissure (FIG. 2A, site #1) or at the foramen rotundum (FIG. 2A, site #2). Without tying the nerve as in the CCI-ION model, the trigeminal inflammatory constriction (TIC) (site #1) nerve injury model and the less invasive foramen rotundum inflammatory compression trigeminal infraorbital nerve (FRICT-ION) (site #2) models were developed to study behavioral responses and molecular mechanisms occurring during transition from acute to chronic craniofacial neuropathic pain (three weeks post injury, TIC) and at longer chronic time points.

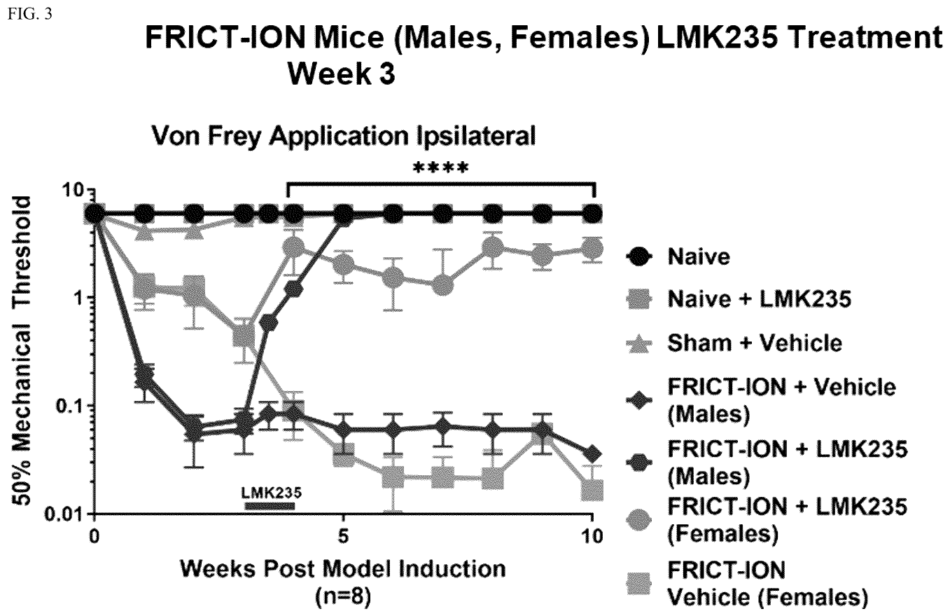

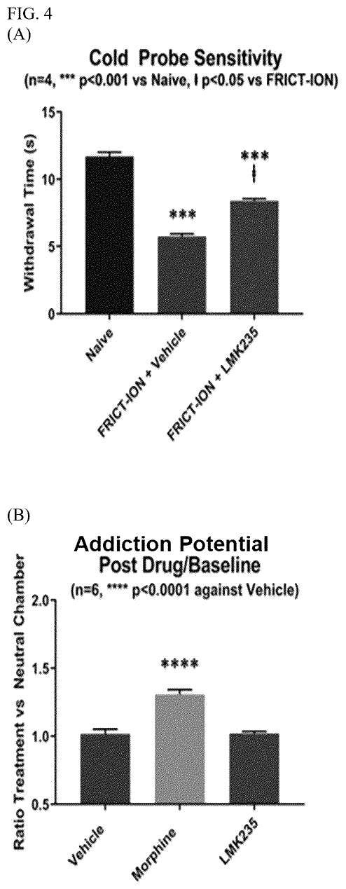

The FRICT-ION model, with its invisible intraoral surgical site, allows study blinding and provides particular ease of induction in less than 10 minutes. Mechanical hypersensitivity develops immediately (FIG. 2B), provides significantly different behavioral responses compared to the naïve and sham groups within weeks (FIG. 2B, p<0.05), and the chronic hypersensitivity persists allowing weekly testing in the ION's receptive field on the whisker pad in the FRICT-ION model (FIG. 3). Responses tested on the ipsilateral side are shown for both male and female mice with FRICT-ION in FIG. 2C. Similar to nerve biopsies from patients, the ION compression caused by aligning the chromic gut suture along the nerve provides persistent irritation but does not cause the severe axonal degeneration seen in tied nerve constriction injury models (CCI, CCI-ION). Cold allodynia reported solely by patients with trigeminal neuralgia is also observed in the FRICT-ION model (FIG. 4A).

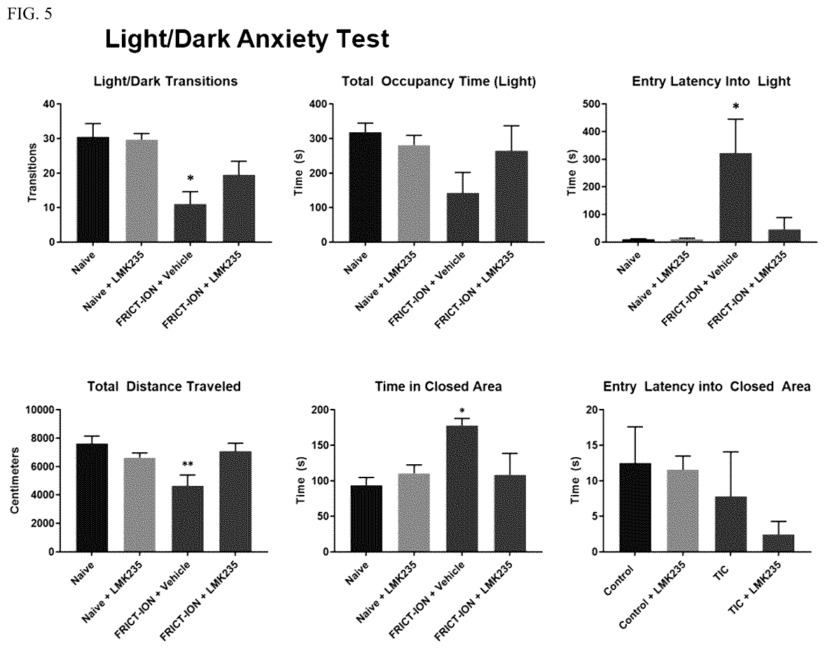

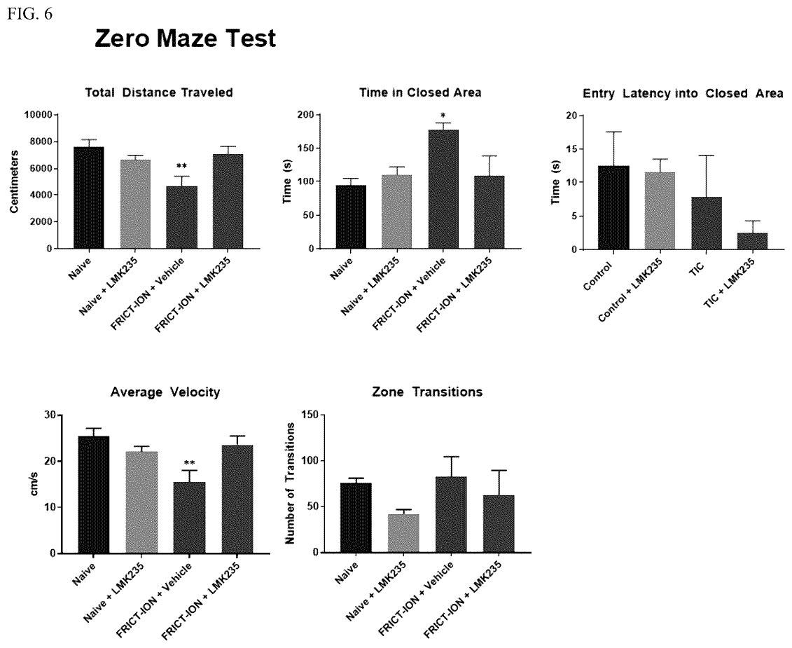

The chronic orofacial neuropathic pain models have a stable time course of mechanical and cold hypersensitivity. The prolonged time course of the model allows studies more relevant to chronic pain including non-evoked emotional responses developing more than 4-6 weeks after model induction (FIG. 5, FIG. 6), persisting in vitro neuronal activation, and epigenetic profile alterations at 10 weeks.

### LMK235, a Class IIa HDAC Inhibitor, Reverses Mechanical and Cold Hypersensitivity Behaviors in Two Mouse Models of Trigeminal Neuropathic Pain

In the current study, the HDACi LMK235 was tested as an in vivo treatment for chronic craniofacial neuropathic pain using the seven daily doses described previously (Trazzi et al., 2016, Hum Mol Genet September 15; 25(18):3887-3907). When pain related behaviors were well established, post-treatments proceeded as follows:
(i) LMK235 Post-treatment Week 3 or Week 8 in C57BL/6 mice with TIC model. Daily subcutaneous injections of LMK235 in week 3 or week 8 after induction of the TIC model in C57BL/6 mice tested on the ipsilateral side when hypersensitivity behaviors plateau (FIG. 2B,C).
(ii) LMK235 Post-treatment Week 3 in BALBc mice with FRICT-ION nerve injury.
   Daily Subcutaneous Injections in Post-Surgical Week 3 in Male and Female BALBc Mice with FRICT-ION Nerve Injury (FIG. 3).

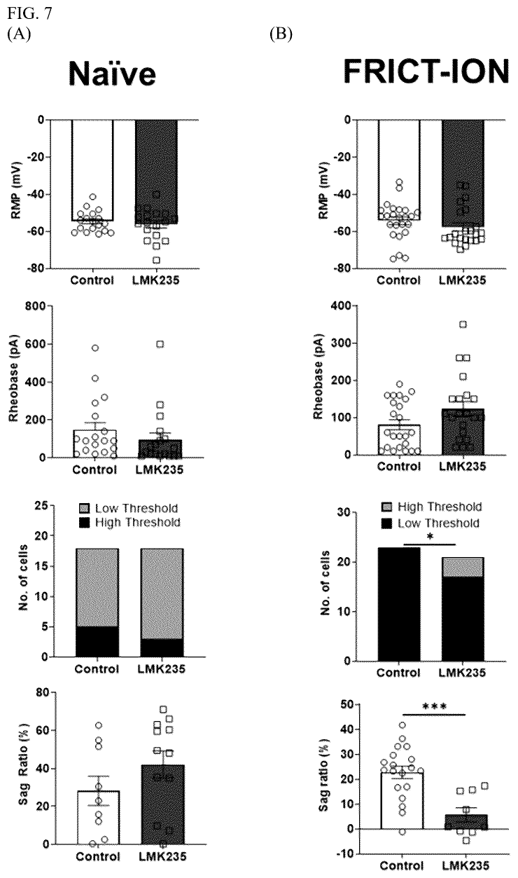

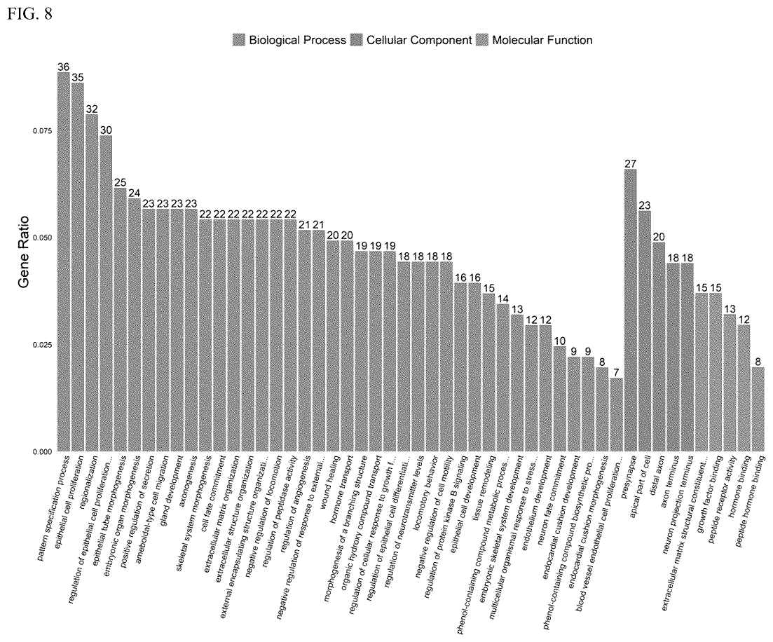

Reflexive mechanical and cold sensitivity (FIG. 4A) were assessed on the whisker pad for group comparisons with this model. Anxiety-related measures were tested only once at the end of the studies (Light/Dark Place Preference, FIG. 5; Zero Maze, FIG. 6) in weeks 6-8. In vitro patch clamp assessments determined TG neuronal responses to LMK235 at the end of week 3 post injury/post-treatment (FIG. 7). At the experiment conclusion in week 10, TG were dissected and evaluated for RNA profiling and HDAC5 protein (FIG. 8-14). Comparisons were made among naïves and mice with TIC or FRICT-ION trigeminal nerve injury, either untreated or post-treated with LMK235 treatment.

LMK235 Post-Treatment Week 3 or 8 in Male C57BL/6 Mice with TIC Model

Post-treatment of the TIC-injury mice for seven days with Class IIa HDAC inhibitor LMK235 (s.c., 5 mg/kg) in the week 3 paradigm returned whisker pad mechanical threshold to naïve baseline where it remained through 4-6 weeks of testing (FIG. 2B). In the second paradigm, post-treatment with LMK235 8 weeks after TIC nerve injury significantly increased the mechanical threshold (FIG. 2C). Testing on the contralateral side indicated a complete return to naïve baseline was provided by LMK235 in the TIC mice treated in week 8 (not shown) ANOVA *p>0.05, ****p<0.001 compared to FRICT-ION+vehicle.

Reversal of Persisting Pain-Related Behaviors with LMK235 Post-Treatment in Male and Female BALBc Mice with FRICT-ION Model

LMK235 reversed von Frey hypersensitivity in both male and female FRICT-ION mice treated daily throughout week 3 (s.c., 5 mg/kg). The week 3 treatment in FRICT-ION mice restored mechanical threshold to baseline where it remained for the subsequent 7 weeks of testing (FIG. 3), [F(11, 168)=45.14] two-way ANOVA *p<0.05, ****p<0.001 compared to untreated mice with TIC or FRICT-ION. In post-hoc analyses, Bonferroni adjustment to all P-values for week-by-week comparisons of FRICT-ION versus Control yields all twelve P-values<0.0012.

Sensitivity to a cold probe (10° C.) applied to the snout in week 8 was reduced by LMK235 in FRICT-ION mice (FIG. 4A), ANOVA ***p>0.001, ****p<0.0001 compared to naïve mice, and p<0.05 compared to FRICT-ION+vehicle.

### Conditioned Place Preference (CPP) Assessment Predicts LMK235 has No Addictive Potential

The conditioned place preference (CPP) test was used to compare morphine and LMK235 post-drug behaviors to baseline. Time spent in their assigned drug administration chamber post-drug was divided by the time spent in the chamber during the baseline recording. A score of 1 meant that there was no difference in time spent in either chamber after dosing. A score greater than 1 meant more time was spent in the assigned drug chamber, and a score less than 1 meant less time was spent in the assigned drug chamber. One-Way ANOVA confirmed that mice given morphine spent more time in their assigned drug chamber, whereas time for mice treated with LMK235 did not vary from baseline indicating no addictive potential (FIG. 4B), ANOVA ****p<0.0001 compared to FRICT-ION+vehicle.

### LMK235 Inhibits Development of Anxiety-Like Behaviors

Anxiety-like, non-evoked measures were tested once to avoid practice effects, in post-surgical weeks 6-9, in the females and in some of the male mice.

Anxiety-like behaviors were assessed using the light/dark box test. Untreated FRICT-ION and TIC model mice developed anxiety-like behavior. LMK235 inhibited the development of anxiety-like behaviors including the (i) number of transitions between chambers, (ii) latency of first re-entry (transition) back into the dark chamber, (iii) number of rearing events, and (iv) number of rearing events in the dark chamber in the TIC model mice (FIG. 5).

High anxiety states are directly related to open area avoidance. Fear/anxiety-like behavior was determined in the zero maze by the (i) number of open and closed entries, (ii) total open and closed area occupancy, and (iii) by the number of exploratory rearing events. All measures were significantly altered in mice with TIC compared to naïve controls indicating significant anxiety, while the behaviors in LMK235-treated mice were not different from naïve mice (FIG. 6), ANOVA *p<0.05, **p<0.01.

LMK235 Reduces the Excitability of TG Neurons from FRICT-ION Mice

The excitability of TG neurons is directly linked to the pathophysiology of trigeminal neuropathic pain. In order to determine the effect of LMK235 on TG neurons from FRICT-ION mice, whole-cell patch-clamp electrophysiological recordings were made from small (<30 μm) TG neurons in the presence of LMK235 (13 μM) or vehicle (0.1% DMSO). TG neurons were obtained from FRICT-ION mice at three weeks post injury or naïve mice. The TG neurons were treated for one hour in vitro with either LMK235 or vehicle control prior to recording (FIG. 7). There were no significant differences observed in resting membrane potential (RMP) or rheobase (current required to elicit firing) between LMK235-treated or vehicle-treated neurons under naïve or injured conditions (p>0.05, Mann-Whitney test). However, a significant difference in the distribution of high and low threshold TG neurons under FRICT-ION (p<0.05, Fisher's exact test, FIG. 7B), but not naïve conditions (FIG. 7A). High threshold neurons were characterized as those having a rheobase of greater than 200 pA, whereas low threshold neurons had a rheobase of less than 200 pA. Approximately 20% of neurons recorded under LMK235-treated conditions were high threshold, whereas none of the neurons recorded under control conditions were high threshold. Unexpectedly, a significant difference in sag ratios also was observed (p<0.001, Mann-Whitney test, FIG. 7B).

The appearance of sag ratio is linked to the activity of hyperpolarization-activated cyclic nucleotide-gated (HCN) channels. The sag ratio of TG neurons from mice with neuropathic pain under LMK235-treated conditions was reduced compared to vehicle-treated controls. In contrast, there was no effect on the sag ratio following LMK235 treatment under naïve conditions. These findings indicate that LMK235 reduces the excitability of TG neurons from FRICT-ION mice.

### Gene Expression Analysis and Alterations in HDAC5, Ion Channels, and Other Genes and Biological Processes

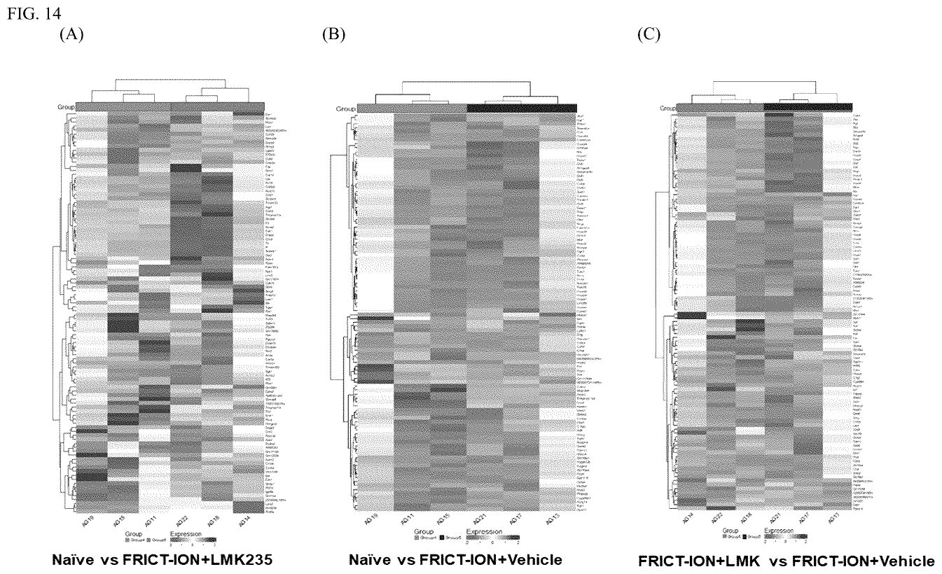

Profiles for ion channel and injury-related gene alterations in TG of LMK235-treated and untreated nerve injured male mice 10 weeks post FRICT-ION nerve compression were compared to naïve controls. RNAseq, GO analyses, heat maps, Volcano plots, and RT-PCR profiles are provided. Genes with at least a two-fold (p<0.05) increase or decrease are identified in the data table analyses, and those with five-fold differences are plotted in the heat maps (FIG. 14).

Over 200 genes were significantly upregulated and >200 were downregulated in FRICT-ION mice compared to naïves. Fewer genes (120) were upregulated in the FRICT-ION mice treated with LMK235 compared to those untreated, and fewer were downregulated (148). Many fewer genes (84 genes) were significantly upregulated or downregulated (68) in FRICT-ION mice treated with LMK235 compared to the naïve group. p<0.05 was considered significant.

Alterations in TG RNA of Mice with FRICT-ION 10 Weeks after Induction Compared to TG of Naïve Mice

Unbiased RNAseq analysis found 172 differentially expressed genes with at least a two-fold change in TG of mice with FRICT-ION 10 weeks after induction compared to TG of naïve mice (p<0.05, Table 1). HDAC5 RNA was increased 2.1-fold (p=2.14×10⁻²) and HDAC9 was increased 1.28-fold (5.57×10⁻⁴). Ion channel and transport genes Kcne2 and P2rx4 were upregulated 11.10-fold (p=1.24×10⁻²) and 2.12-fold (p=1.28×10⁻²). Transcription factor associated genes were upregulated as follows: Hoxc8 was upregulated 25.26-fold (p=4.63×10⁻⁵), Hoxb9 was upregulated 11.88-fold (p=1.73×10⁻⁷), and Hoxd8 was upregulated 5.99-fold (p=6.46×10⁻⁸). Nerve regeneration associated genes were upregulated as follows: Ttr was upregulated 12.72-foldfold (p=5.36×10⁻⁴) and Folr1 was upregulated 6.65-fold (p=3.28×10⁻²). The growth hormone gene Gh was upregulated 8.06-fold (p=4.46×10⁻³) 10 weeks after nerve injury. Remyelination-associated gene Sostdc1 was upregulated by 7.93-fold (p=1.03×1002). The interferon-α-inducible macrophage gene Slfn4 activated by Toll-like receptor agonists (GeneCard) was upregulated 2.72-fold (p=2.25×10⁻²). Hypothalamic-pituitary-adrenal (HPA) axis-related genes were downregulated as follows: Crhbp was downregulated 2.51-fold (p=7.76×10⁻⁸) and Tph2 was downregulated and 4.51-fold (p=1.27×10⁻⁷). Excitatory synapse formation associated gene C1q14 was downregulated 5.44-fold (p=7.65×10⁻⁶) after 10 weeks of FRICT-ION model induction.

### GO Analysis for TG of Male FRICT-ION Mice Compared to Naïves

GO analysis in TG of male FRICT-ION mice compared to naïves (FIG. 8) showed biological processes included axonogenesis (Gbx1/Otx2/Robo3), wound healing (C3/Cd44/Cldn1/F2rl2/Il1a/S100a9/Thbs1), hormone transport (Crhbp/Nmu/Ttr), regulation of neurotransmitter levels (Moxd1/Sic6a4), and tissue remodeling (Il1a/II20ra). Additional biological processes not shown on the graph included regulation of inflammatory response (S100a9, S100a8), regulation of wound healing (S100a9), chronic inflammatory response (S100a9), and sensory perception of pain (Nmu). Molecular functions included growth factor binding (Kl/Thbs1), hormone binding (Crhbp/Glp2r/Oxtr/Ttr), and peptide hormone binding (Crhbp/Glp2r/Oxtr). Molecular functions not shown on the graph included hormone activity (Ttr).

Alterations in TG of LMK235-Treated Mice with FRICT-ION 10 Weeks after Induction Compared to TG of Naïve Mice

Data comparing LMK235-treated FRICT-ION mice to TG of naïve mice are shown in Table 2. Fifty-three genes were differentially expressed with at least a two-fold change (p<0.05) in mice treated with HDAC5 inhibitor, LMK235, compared to naïve mice. Nerve regeneration associated genes were upregulated as follows: Ttr was upregulated by 23.21-fold (p=3.98×10⁻⁵) and Folr1 was upregulated 11.92-fold (p=6.97×10⁻³). Ion channel and transport genes were upregulated as follows: Kcnj13 was upregulated 7.84-fold (p=0.01), Kcne2 was upregulated 17.29-fold (p=4.00×10⁻³), and Clic was upregulated 64.92-fold (p=1.28×10⁻²). The remyelination-associated gene Sostdc1 was upregulated by 15.70-fold (p=1.08×10⁻³) at 10 weeks for LMK235-treated male FRICT-ION mice.

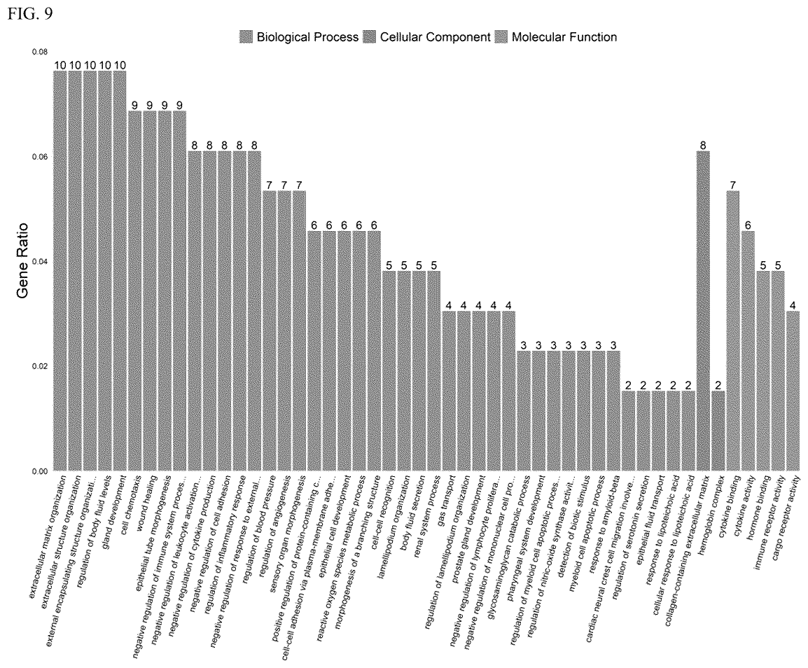

GO Analysis for male FRICT-ION mice treated with HDAC5 inhibitor, LMK235, compared to naïves GO analysis for male FRICT-ION mice treated with HDAC5 inhibitor, LMK235, compared to naïve controls (FIG. 9) showed biological processes included wound healing (Aqp1/Cidn1/F5), negative regulation of immune system process, negative regulation of leukocyte activation, negative regulation of cytokine production (Lbp), and regulation of inflammatory response (Lbp). Biological processes not shown on the graph included hormone transport (Ttr), axon regeneration (Folr1), and response to axon injury (Folr1). Molecular functions included cytokine binding (Ackr4/Sostdc1), cytokine activity (Tnfsf13), hormone binding (Crhr2/Ttr), and immune receptor activity (Ackr4).

Alterations in TG of LMK235-Treated Mice with FRICT-ION 10 Weeks after Induction Compared to TG of FRICT-ION Mice Treated with Vehicle

Table 3 shows data directly comparing LMK235-treated FRICT-ION mice to TG of FRICT-ION mice treated with vehicle. Ninety-seven genes were differentially expressed with at least a two-fold change (p<0.05) in mice treated with HDAC5 inhibitor, LMK235, compared to mice treated with vehicle. Histone associated gene Hist2h3c2 was upregulated by 128.58-fold (p=3.02×10⁻³). Excitatory synapse formation associated gene C1q14 was upregulated 4.76-fold (p=3.54×10⁻⁵). Hypothalamic-pituitary-adrenal (HPA) axis-related genes were upregulated as follows: Tph2 was upregulated 4.18-fold (p=4.82×10⁻⁷) and Crhbp was upregulated 2.79-fold (p=1.62×10⁻⁹). Transcription factor related genes were downregulated as follows: Hoxd8 was downregulated 4.14-fold (p=1.02×10⁻⁵), Hoxc8 was downregulated 8.22-fold (p=2.32×10⁻³), and Hoxb9 was downregulated by 16.50-fold (p=7.21×10⁻⁹). The growth hormone gene Gh was downregulated 25.22-fold (p=9.19×10⁻⁵). Ion transport related genes were down regulated as follows: P2rx4 was downregulated 2.08-fold (p=3.31×10⁻²) and ribonuclease activity associated gene Slfn4 was downregulated 2.32-fold (p=4.63×10⁻²).

GO Analysis for Male FRICT-ION Mice Treated with HDAC5 Inhibitor, LMK235, Compared to FRICT-ION Mice Treated with Vehicle

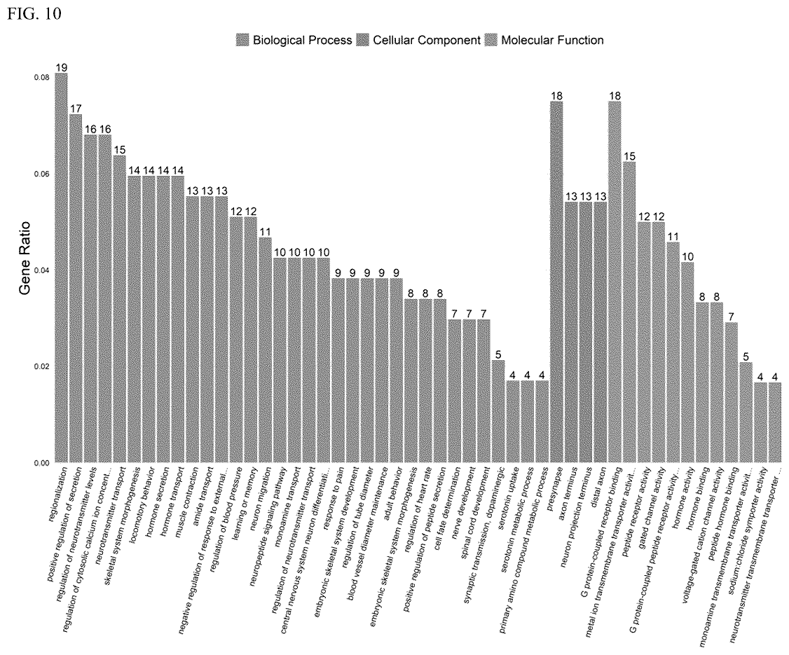

The GO analysis for male FRICT-ION mice treated with, LMK235, compared to FRICT-ION mice treated with Vehicle (FIG. 10) included biological processes such as regulation of neurotransmitter levels (Fev/Slc18a2/Slc6a2/Slc6a4), neurotransmitter transport (Fev/Slc18a2/Slc6a2/Slc6a4), hormone secretion (Crhbp/Gata3/Isl1/Pde4c/Sic18a2), hormone transport (Crhbp/Gata3 Isl1/Pde4c/Sic18a2), neuron migration (Gata3), neuropeptide signaling pathway, monoamine transport (Fev/Slc18a2/Slc6a2/Slc6a4), regulation of neurotransmitter transport (Fev/Slc18a2), central nervous system neuron differentiation (Isl1), response to pain (Slc6a2), and synaptic transmission: dopaminergic (Crhbp/Sic6a2/Slc6a4). Biological processes not shown on the graph included regulation of neuron differentiation (Slc6a4). Molecular functions included hormone activity, hormone binding (Crhbp), and peptide hormone binding (Crhbp).

### Comparison of Changes in Gene Expression Among all Groups

A two-fold increased expression of HDAC5 RNA and protein was found in RNAseq and Western blot data following trigeminal nerve injury. The significant increase in HDAC5 RNA and protein was not found in the mice treated with the selective Class IIa HDAC4/5 inhibitor LMK235. Strikingly, while HDAC5 was increased 2.1-fold (p=2.14×10⁻²) in the FRICT-ION mice (Table 1), the Table 3 shows there was a −2.03-fold (p=4.95×10⁻²) difference in LMK235-treated versus untreated mice. This negation of the HDAC5 increase by LMK235 is reflected in the effective reversal of the pain-related behaviors induced by the nerve injury. In fact, the HDAC5 (HdacS; −1.13-fold, p=0.003) and HDAC10 (Hdac10; −1.20-fold, p=0.05) in LMK235 treated mice are decreased below the naïve controls (Table 2). No HDAC4 was evident in TG RNA profiles for any of the group screens at 10 weeks. Thus, while it is known nerve injury enhances direct modification of histones through HDACs ability to inhibit gene transcription (FIG. 1), the actions of HDACi LMK235 effectively diminished post-injury increases in HDAC5 protein and HDAC5 RNA, as well as promoted or inhibited most other observed gene alterations. The findings indicate HDAC5 is an active component in neuronal activation and pain-related behavior in a chronic trigeminal nerve injury neuropathic pain model.

Table 6 provides detailed information for mouse genes up- and down-regulated that are also reported in human trigeminal neuralgia. Detailed gene profiling of TG after LMK235 treatment of mice with FRICT-ION chronic nerve injury was strikingly similar to that of naïve mice in week 10 in contrast to that of untreated FRICT-ION mice with chronic neuropathic pain. Reversal of behavioral hypersensitivity threshold and trigeminal neuron excitability with Class IIa HDACi LMK235, as well as the increased HDAC5 RNA and protein levels in nerve injured TG provide strong data corroborating HDAC5 epigenetic regulation of craniofacial neuropathic pain. Importantly, the molecular profiles examined during the more clinically relevant chronic pain phase at 10 weeks post nerve injury indicate the ability of LMK235 to diminish cytokines and increase neuronal repair mechanisms that contribute to chronic neuropathic pain.

The first column of Table 7 provides genes that are regulators of nerve regeneration. These genes initiate reprograming events that allow differentiation of cells to enter a state amenable to tissue and nerve repair. The mechanisms and pathways by which the regeneration can occur is shown in the second column. These genes identified in week 7 after nerve injury indicate transcriptomic changes favoring nerve regeneration account for the durable reversal of pain by LMK235. Significant changes are noted with an asterisk. Genes up-regulated or down-regulated in the FRICT-ION pain model compared to naïve male mice are provided in the third column. The ability of epigenetic modulator LMK235 to suppress and activate cellular and molecular events with HDAC5 inhibition indicate nerve repair is responsible for the durable pain reversal.

Several other RNAs altered in the FRICT-ION model with only vehicle treatment were returned back toward naïve baseline by LMK235. Complement component 1, q subcomponent-like 4 (C1q14) with its TNF-like structure was downregulated 5.44-fold in the FRICT-ION mice, but levels were not significantly different from naïve mice with the HDAC5 inhibitor treatment. Corticotropin releasing hormone binding protein (Crhbp), associated with stress and depression, while downregulated 2.5-fold in FRICT-ION, was not significantly different form naïve controls in FRICT-ION mice treated with LMK235. Increased P2rx4 RNA in mice with FRICT-ION (2.12-fold) is not different from naïves if mice with FRICT-ION are treated with the HDAC5 inhibitor. Schlafen (Slfn4) RNA increased in FRICT-ION model mice (2.72-fold), is not different from naïve controls if FRICT-ION mice are treated with the LMK235. Similar increases in schlafen RNA are found in all other profiles published for nerve injury models.

In Table 4, differential gene expression from GO analyses are presented as percentage of differentially expressed genes in TG from male FRICT-ION mice given daily LMK235 or Vehicle, compared to naïve mice and with each other. This presentation of the gene ontology is useful in better interpreting the high throughput molecular data and providing detail about the underlying biological phenomena of the study. Table 4 functional analysis shows that for differentially expressed genes in TG of male FRICT-ION mice compared to naïve mice 8% of the genes are involved with wound healing, 0.5% are involved with the chronic inflammatory response, 2% are involved with serotonin secretion and metabolic process, and 3% are involved with negative regulation of cytokine production.

For differentially expressed genes in TG of male FRICT-ION mice treated with LMK235 compared to naïve, 11% of the genes were involved with wound healing, 3% were involved with serotonin transport and secretion, and 6% were involved with negative regulation of cytokine production.

For the differentially expressed genes in TG of male FRICT-ION mice treated with LMK235 compared to FRICT-ION mice treated with vehicle, 8% of the genes were involved with wound healing, 2% were involved with negative regulation of the inflammatory response, 5% were involved with learning or memory, and 3% were involved with serotonin uptake and metabolic process.

### Ion Channel Genes

The following ion channel genes were upregulated in LMK235-treated FRICT-ION mice compared to untreated FRICT-ION mice: Scn8a (1.05-fold, p=1.98×10⁻¹), Kcnk1 (1.05-fold, p=2.82×10⁻¹), Scn9a (1.29-fold, p=2.62×10⁻⁴), Trpc6 (1.08-fold, p=6.17×10⁻¹), Trpm3 (1.19-fold, p=2.47×10⁻²), Trpm4 (1.05-fold, p=6.82×10⁻¹), and Clcn2 (1.01-fold, p=8.92×10⁻¹). Ion channels downregulated in LMK235-treated FRICTION mice compared to untreated FRICT-ION mice were Cacna1h (1.49-fold, p=2.21×10⁻⁵), Scn2a Scn2a1 (1.04-fold, p=3.85×10⁻¹), Scn3a (1.01-fold, p=9.15×10⁻¹), Scn7a (1.07-fold, p=6.29×10⁻¹), Trpv6 (1.19-fold, p=3.74×10⁻¹), Kcna5 (1.41-fold, p=1.33×10⁻²), Kcnd2 (1.00-fold, p=9.66×10⁻¹), Kcnh7 (1.19-fold, p=2.36×10⁻¹), Kcnj6 (1.38-fold, p=2.75×10⁻²), Kcns2 (1.25-fold, p=5.91×10⁻²), Cacna1d (1.04-fold, p=5.84×10⁻¹), Cacna1g (1.43-fold, p=3.12×10⁻⁴), Cacna1 (1.39-fold, p=4.03×10⁻⁶), and CacnbI (1.13-fold, p=1.13×10⁻²). Ion channel gene Kcne2 was upregulated 11.10-fold (p=1.24×10⁻²) in FRICT-ION mice compared to naïve and greatly but insignificantly reduced (1.56-fold, p=6.10×10⁻¹) in LMK235-treated mice compared to untreated FRICT-ION mice. Cacna1a, encoding a calcium channel, was upregulated 1.10-fold (p=6.89×10⁻²) in FRICT-ION mice compared to naïve mice while it was downregulated 1.16-fold (p=3.24×10⁻³) in LMK235-treated FRICT-ION mice compared to untreated FRICT-ION mice.

### Pain-Related Genes

Several pain-related genes (P2rx4, Cckbr, growth hormone (Gh), and schlafen (Slfn4)) upregulated in the trigeminal nerve injury FRICT-ION model are diminished to naïve or below naïve levels by the LMK235 by week 7. Upregulation of P2rx4 RNA at 10 weeks after trigeminal nerve compression injury (2.12-fold, p=1.28×10⁻²) was not observed in mice treated with LMK235 3 weeks after injury. Contrarily, LMK235 downregulated P2rx4 RNA expression. Surprisingly, there was only minimal and insignificant foldchanges for P2rx7 in LMK235-treated and untreated FRICT-ION mice. The most prominent pain-related genes altered in the FRICT-ION mice with mechanical hypersensitivity included significant decreases in inhibitory pain neurotransmitter serotonin related RNA for serotonin transporter (Slc6a4; −4.86-fold, p=4.23×10⁻⁶) and serotonin synthetic enzyme tryptophan hydroxylase 2 (Tph2; −4.51-fold, p=1.27×10⁻⁷). The Slc6a4 was upregulated by an equal amount in the LMK235-treated mice.

This clearly implies a decrease in serotonin function and aligns with the FRICT-ION-induced hypersensitivity. Neuropathic pain related genes upregulated in LMK235-treated FRICT-ION mice compared to untreated FRICT-ION mice were Net1 (1.12-fold, p=1.68×10⁻¹), Nrg2 (1.10-fold, p=4.16×10⁻¹), Nova1 (1.04-fold, p=3.01×10⁻¹), Nf2 (1.04-fold, p=2.22×10⁻¹), and Neurod4 (1.57-fold, p=1.47×10⁻¹). Downregulated neuropathic pain related genes in LMK235-treated FRICT-ION mice compared to untreated FRICT-ION mice were Nras (1.03-fold, p=5.21×10⁻¹), Ngef(1.34-fold, p=7.33×10⁻⁷), Nrg3 (1.00-fold, p=9.77×10⁻¹), Nav3 (1.19-fold, p=2.68×10⁻²), Nts (1.74-fold, p=4.23×10⁻⁷), and Nkain2 (1.14-fold, p=5.34×10⁻²).

### Somatostatin

Genes for somatostatin and its receptors were not drastically altered in either untreated or treated FRICT-ION mice.

### Ntrk1

Ntrk1, encoding a nerve growth factor receptor, was upregulated 1.80-fold (p=9.90×10⁻⁴) in LMK235-treated FRICT-ION mice compared to untreated FRICT-ION mice.

### Mpz

Mpz was downregulated 1.05-fold (p=9.47×10⁻¹) in LMK235-treated FRICT-ION mice compared to untreated FRICT-ION mice.

### Maoa

Maoa was upregulated 1.14-fold (p=2.64×10⁻³) in LMK235-treated FRICT-ION mice compared to untreated FRICT-ION mice.

### Bone Morphogenetic Genes

Bmp2, Bmp3, and Bmp5 were all downregulated 1.02-fold (p=8.75×10⁻¹), 2.70-fold (p=7.00×10⁻⁵), and 1.01-fold (p=9.64×10⁻¹) in LMK235-treated FRICT-ION mice compared to untreated FRICT-ION mice respectively. Bmp4 was not affected.

### Growth Hormone

Growth hormone (Gh) RNA was increased in the TG of FRICT-ION mice (8.06-fold, p=4.46×10⁻³) compared to naïve mice but greatly decreased in LMK235-treated mice (25.22-fold, p=9.19×10⁻⁵) compared to untreated FRICT-ION mice.

Heat Maps of the Pain Related Genes from the GO Analyses

The GO and heatmap analyses provide insight into potential mechanisms for LMK235's ability to reverse the behavioral and neuronal recording indicators of chronic neuropathic pain. The heat maps show the relationship of differentially expressed genes with rows representing the genes differentially expressed and the columns representing comparison groups (FIG. 14). Only genes with two-to-five-fold changes (p<0.05) and a known gene ontology were included in the heat map. The genes were grouped into eight categories for the heat map most relevant to pain which included wound healing, transcription/regulation of gene expression, pain, neurogenesis/neuron differentiation/axogenesis/axon regeneration/cell division, learning or memory, ion channel/ion binding/ion transport, immune/inflammatory/cytokine/hormone/neurotransmitter, and behavioral response.

The comparison of LMK235-treated FRICT-ION group with naïves displayed a notable lack of genes altered. The largest collections of pain related genes were the immune/inflammatory/cytokine/hormone/neurotransmitter grouping and the transcription/regulation of genes grouping, including numerous sequence-specific DNA binding terms (Hoxc8, Hoxb9, Hoxd8, Hoxd9, Hoxa6, Hoxb8, Hoxb7, Hoxc6, Hoxb6). Neurogenesis/neuron differentiation/axogenesis/axon regeneration/cell division/wound healing/myelin repair/ion channel genes were also prominent.

The impact of macrophages, satellite glia, and certain cytokines on neuronal damage and repair is well known. Peripheral nerves possess self-repair capabilities, but those with marked damage or substantial defects are challenging to repair extrinsically. Central nervous system nerves have no or limited repair capacity. Investigating the pathophysiology of peripheral nerve repair is important for the clinical treatment of peripheral nerve restoration and regeneration.

RNA profiles and GO analyses indicate axonal repair is a factor in nerve recovery following injury. The remyelination-associated gene Sostdc1 is upregulated by Class IIa histone deacetylase and mediates in synaptic plasticity, axon regeneration, neurite extension, and neural differentiation. Sostdc1 is highly expressed in the developing optic fiber layer, optic nerve, and ganglion cells of the human eye and its gene expression was proposed as a biomarker for rheumatoid arthritis. HDAC5 plays a role in regulating gene transcription involved in inhibiting neurite elongation, cellular/system adaptations to chronic emotional stimuli, and drug-induced circuitry changes. Nuclear export of HDAC5 by the back-propagated calcium influx after nerve injury is followed by its transport to damaged nerve endings, where it promotes nerve regeneration and neuronal plasticity. HDAC5-deacetylated tubulin in microtubules following peripheral sciatic nerve ligation promotes growth cone formation for axon regeneration. In contrast, tubulin deacetylation did not occur in ligated optic nerves or DRG from mice with hemisected spinal cords, demonstrating neuronal regeneration via injury-induced tubulin deacetylation is limited to the peripheral nervous system. In another model of nerve injury, model-specific Hdac6 genetic deletion from sensory neurons did not avert cisplatin-induced mechanical hypersensitivity, while global knockout of HDAC6 was protective. This was interpreted to signify a role of HDAC6 in other cell types since depletion of MRC1 (CD206)-positive macrophages locally decreased the ability of an HDAC6 inhibitor to reverse cisplatin-induced mechanical allodynia. While microglia were not affected, M2-macrophage-dependent spinal cord I1-10 mRNA and signaling were increased with the HDAC6 inhibitor. Other genes with large differences by group (>5-fold) were not shown on the heat maps but are included in Table 5.

### Volcano Plots

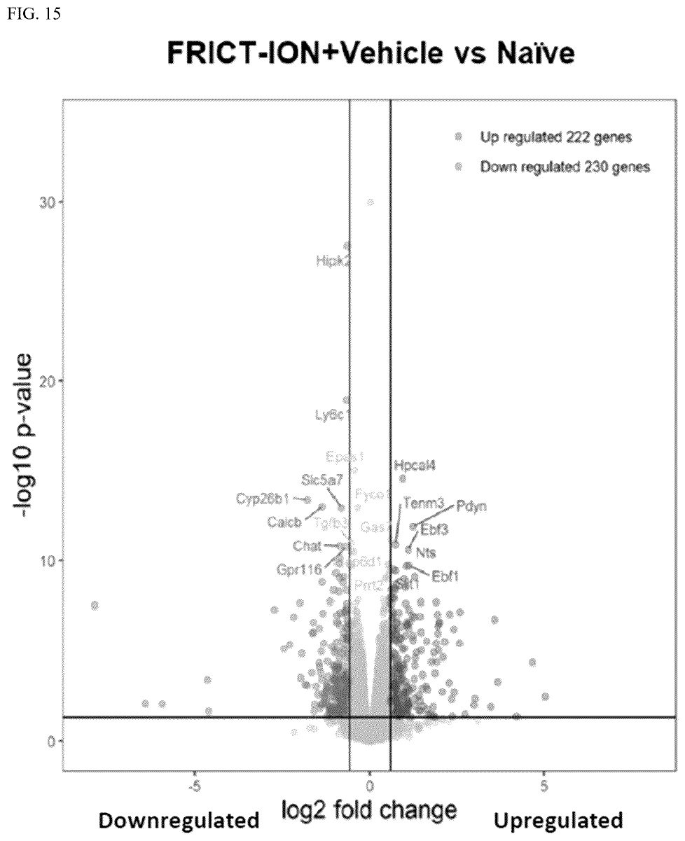

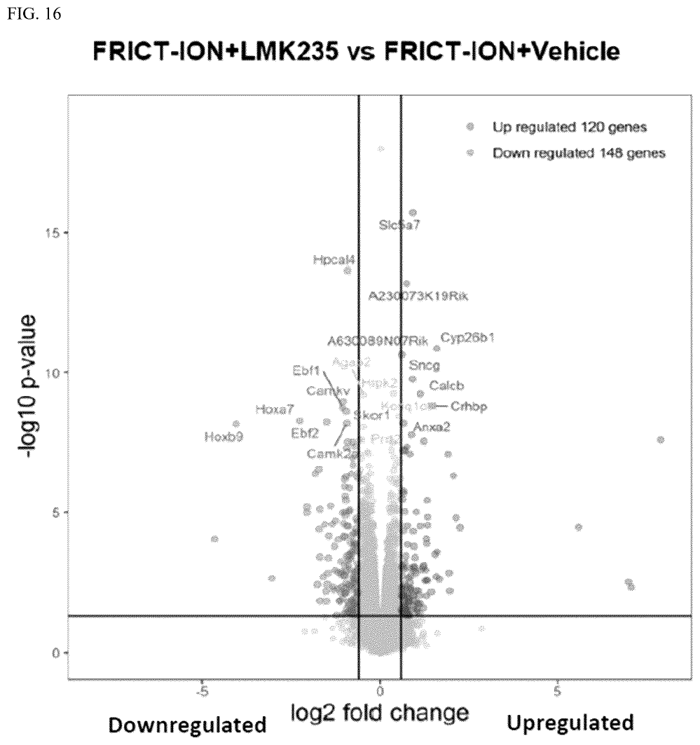

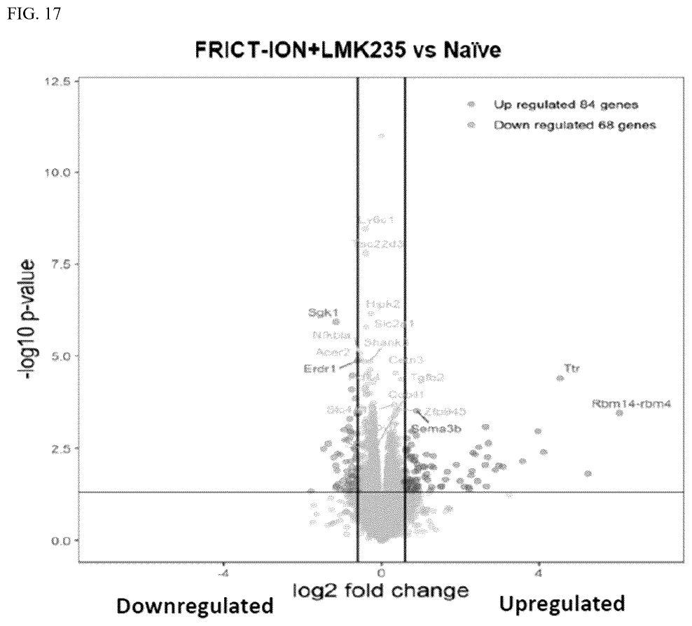

FIGS. 15-17 provide volcano plot representations of the two-fold differences present the RNAseq analysis from male mice comparing each of the test groups to the naïves and to each other (naïve, untreated FRICT-ION mice, FRICT-ION mice treated with LMK235). While 222 upregulated and 230 downregulated genes were found in the TG of FRICT-ION mice with hypersensitivity at 10 weeks post injury compared to naïve mice, many fewer genes were found in the other comparisons. For example, differences in RNA expression were minimal in the comparison between LMK235-treated mice with FRICT-ION and naïve mice with only 84 genes upregulated and 68 genes downregulated.

### RT-PCR

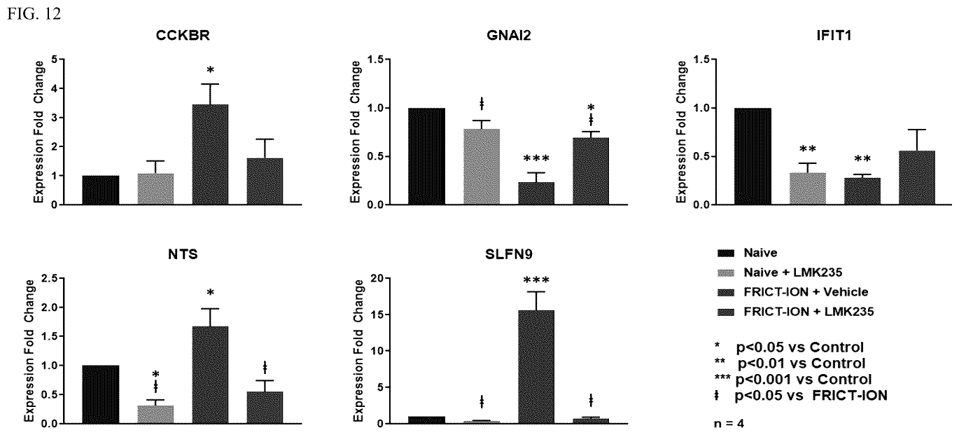

To confirm that the anti-allodynic mechanism provided by LMK235 was accompanied by changes in RNA of specific pain relate genes, TG expression of several genes was assessed in the TG of LMK235-treated and untreated FRICT-ION groups to naïve controls with RT-PCR (FIG. 12).

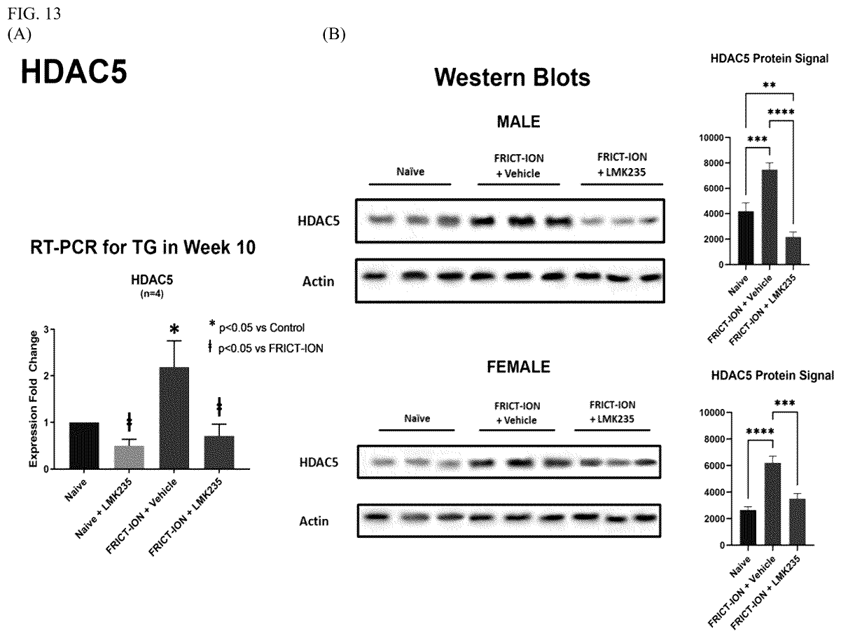

The RNAs for cholecystokinin B receptor (CCKBR), neurotensin (NTS), and schlafen 9 (SCFN9) were significantly increased in the FRICT-ION mice but normalized by LMK235 treatment. The GNAI2 and IFIT1 were decreased in FRICT-ION mice and partially restored in the LMK235-treated mice with FRICT-ION. The TG isolated from FRICT-ION mice at 10 weeks post injury had upregulated genes encoding HDAC5 (Hdac5; two-fold, p=0.04) (FIG. 13A), HDAC10 (Hdac10; 1.2-fold, p=0.04) and HDAC11 (Hdac11; 1.2-fold, p=0.02) compared to controls. LMK235 reduced HDAC5 expression in both naïve controls (p<0.05) and FRICT-ION mice (p<0.05), but changes in HDAC10 or HDAC11 genes in LMK235-treated naïve mice were not observed. Rather, changes in the following histone-related genes were observed in LMK235-treated FRICT-ION mice: histone cluster 1, H2bc (Hist1h2bc; 1.4-fold, p=0.004) and histone cluster 2, H2aa1 (Hist2h2aa1; 1.6-fold, p=0.02).

### RNA Microarray (C57B1/6 Mice)

Isolated RNA from TG of male C57B1/6 mice three weeks post TIC injury was hybridized to the Mouse Gene 2.0 Array. RNA gene chip microarray studies show upregulation of genes encoding HDAC5 (Hdac5; 1.9-fold, p=0.02), HDAC10 (Hdac10; 1.3-fold, p=0.0008) and HDAC11; (Hdac11 1.2-fold, p=0.03) compared to controls. This was almost identical to the RNAseq profile at 10 weeks, indicating the HDAC RNA increase likely persists continuously in weeks 3-10. Changes in histone-related gene histone cluster 2; H2aa1 (Hist2h2aa1; 2.4-fold, p=0.02) was observed in TG from TIC mice compared to controls.

### HDAC5 Protein Levels are Differentially Altered in LMK235-Treated and Untreated Male and Female FRICT-ION Mice Compared to Controls

In order to determine whether HDAC5 protein levels were altered in the TG, Western blot experiments were performed in both male and female mice. TG HDAC5 protein levels were increased in FRICT-ION male (ANOVA, p<0.001) and female mice (ANOVA, p<0.0001) compared to naïve controls (FIG. 13B). In addition, LMK235 treatment significantly reduced TG HDAC5 protein levels to naïve levels in females ANOVA, (p<0.001) or below naïve levels in males (ANOVA, p<0.0001).

This disclosure therefore reports the in vivo and in vitro effects of the Class II HDAC inhibitor, LMK235, during the clinically relevant chronic phase of orofacial trigeminal neuropathic pain. Post-treatment with LMK235 not only provided attenuation of craniofacial mechanical and cold hypersensitivity established three weeks prior to treatment, but it also alleviated the non-evoked anxiety-like behaviors that accompany long-term hypersensitivity models. Alleviation of the mechanical hypersensitivity by LMK235 persisted through at least seven subsequent weeks in the FRICT-ION model. In vivo effects of HDACi LMK235 on pain-related behaviors were similar in both the CCI-ION and FRICT-ION models of trigeminal neuropathic pain. The persistent and durable effects after post-treatment were demonstrated in both C57B1k6 and BALBc mouse strains. Thus, LMK235 is not only effective in alleviating mechanical allodynia but also in alleviating development of anxiety-like behaviors associated with chronic pain models. Thus, LMK235 may reduce major clinical complaints of patients with chronic trigeminal neuropathic pain.

Examination of the mechanism of action of LMK235 using electrophysiological analyses established that LMK235 reduced trigeminal neuron excitability. The in vitro characterization profile included a shift where ˜20% of the small neurons recorded under LMK235-treated conditions were high threshold, whereas none of the neurons under control conditions have a high threshold.

Reversal of hypersensitivity and trigeminal neuron excitability with Class II HDACi LMK235, as well as the increased HDAC5 RNA and protein levels provide strong support for HDAC5 epigenetic regulation of craniofacial neuropathic pain. The molecular profiles examined during the more clinically relevant chronic pain phase 10 weeks post nerve injury indicated the ability of LMK235 to diminish expression of cytokines and genes that contribute to chronic neuropathic pain, while concurrently increasing RNA for neuronal repair mechanisms.

No previous HDAC inhibitor post-treatment study has alleviated hypersensitivity persisting 6-12 weeks in a clinically relevant chronic neuropathic pain model. Post-treatment with Class IIa HDACi LMK235 at three weeks post TIC nerve injury effectively reversed hypersensitivity and effectiveness persisted long-term. The HDACi post-treatment eight weeks is the first evidence of diminished mechanical hypersensitivity at such a long-term time point post chronic neuropathic pain. These data suggest that continuing HDACi LMK235 treatment may have provided more complete recovery.

The most prominent pain-related genes altered in the FRICT-ION mice with mechanical hypersensitivity included significant decreases in inhibitory pain neurotransmitter serotonin related RNA for serotonin transporter (Slc6a4; −4.86-fold, p=4.23×10⁻⁶) and serotonin synthetic enzyme tryptophan hydroxylase 2 (Tph2; −4.51-fold, p=1.27×10⁻⁷). The Slc6a4 was upregulated by an equal amount in the LMK235-treated mice. This clearly implies a decrease in serotonin function and aligns with the FRICT-ION-induced hypersensitivity.

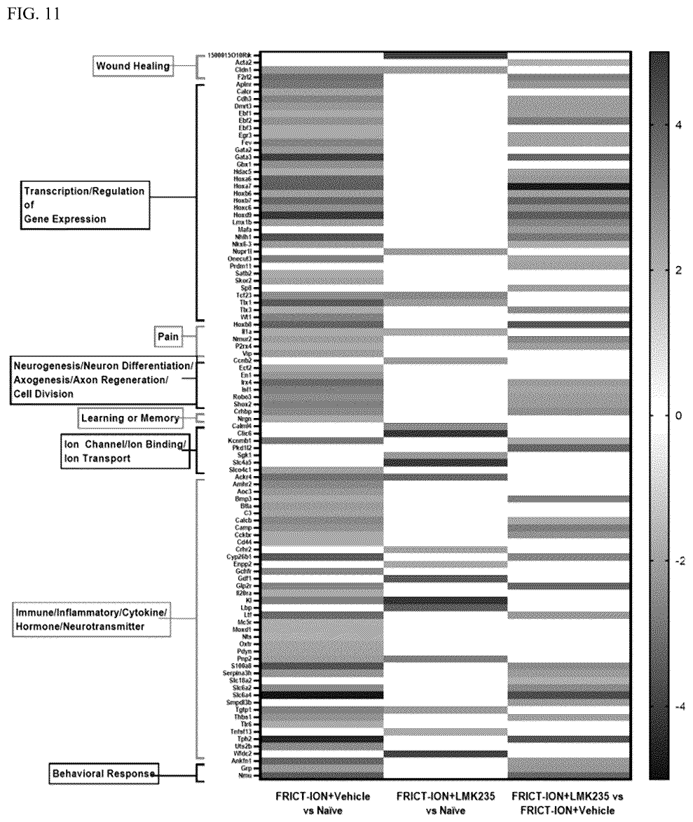

The GO analyses, supportive Table 4, and the heat map analyses (FIG. 11) provide insight into potential indicia for LMK235's ability to reverse the behavioral and neuronal recording indicators of chronic neuropathic pain. Prominent are wound healing genes, transcription factors including numerous sequence-specific DNA binding terms (Hoxc8, Hoxb9, Hoxd8, Hoxd9, Hoxa6, Hoxb8, Hoxb7, Hoxc6, Hoxb6), neuronal/myelin repair, ion channel, and immune/cytokine genes.

Thus, this disclosure described methods for treating neuropathic pain in a subject. Generally, the method includes administering to the subject an amount of the HDAC5 inhibitor, LMK235, effective to alleviate neuropathic pain in the subject. As used herein, the term “treat” or variations thereof refer to reducing, limiting progression, ameliorating, or resolving, to any extent, neuropathic pain. Generally, treatment of neuropathic pain is initiated after the neuropathic pain manifests in a subject. As used herein, an “effective amount” is an amount effective to reduce, limit progression, ameliorate, or resolve, to any extent, neuropathic pain experienced by the subject.

While described herein in the context of exemplary embodiments in which LMK235 is administered to a subject to treat neuropathic pain in a subject, LMK235 may be administered to a subject having or at risk of having, a condition that involves expression (e.g., overexpression) of histone deacetylase 5 (HDAC5) or is characterized at least in part by expression (e.g., overexpression) of HDAC5. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is often initiated before a condition manifests in a subject.

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition—is referred to herein as treatment of a subject that is “at risk” of having the condition. As used herein, the term “at risk” refers to a subject that may or may not actually possess the described risk. Thus, for example, a subject “at risk” of a condition is a subject possessing one or more risk factors associated with the condition such as, for example, genetic predisposition, ancestry, age, sex, geographical location, lifestyle, or medical history. Treatment may also be continued after symptoms have resolved, for example to delay or reduce the likelihood of recurrence.

Accordingly, a composition can be administered before, during, or after the subject first exhibits a symptom or clinical sign of the condition or, in the case of infectious conditions, before, during, or after the subject first comes in contact with the infectious agent. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a particular condition. In this aspect, an “effective amount” is an amount effective to reduce, limit progression, ameliorate, or resolve, to any extent, a symptom or clinical sign related to the condition.

Exemplary conditions that involve HDAC5 expression include, but are not limited to, diabetic neuropathy, inflammatory arthritis, myelofibrosis, periodontitis, osteoporosis, osteopetrosis, bacterial-induced osteolysis, nerve injury, brain injury, systemic sclerosis, rheumatoid arthritis, diabetic kidney disease, acute kidney injury, polycystic kidney disease, polycystic ovary, or an inflammatory gastrointestinal syndrome (e.g., inflammatory bowel disease, short gut syndrome, ulcers, Crohn's disease, or intestinal sepsis), anxiety, depression, or cancer. Exemplary cancers include those associated with Sox2 expression (e.g., breast cancer, lung cancer, esophagus cancer, colon cancer, prostate cancer, ovarian cancer, etc.), overexpression of Lin28b (e.g., breast cancer, hepatocellular carcinoma, colorectal cancer, neuroblastoma, ovarian cancer, head and neck cancer, etc.), expression of c-myc (e.g., breast cancer, etc.), and cancers in which Klf4 acts as an oncogene (e.g., breast cancer, head and neck cancer, glioblastoma, etc.). In one or more embodiments, LMK235 can be used as an adjuvant to improve effectiveness of cancer therapies including, but not limited to, therapies for glioblastoma, lymphoma, or multiple myeloma. Further, LMK235 may improve effectiveness of therapies involving tarnoxifen and/or an aromatase inhibitor by at least partially reversing resistance to tamoxifen, aromatase inhibitor therapy, or both. In one or more embodiments, administering LMK235 to the subject improves cardiac dysfunction, pathological ventricular remodeling, or both.

The subject can be a human or a non-human animal such as, for example, a livestock animal, a laboratory animal, or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, or reindeer), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

LMK235 may be formulated with a pharmaceutically acceptable carrier. As used herein, “carrier” includes any solvent, dispersion medium, vehicle, coating (including but not limited to nanoparticle coating for sustained delivery), diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with LMK235, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with LMK235 without causing any undesirable biological effects or interacting in a deleterious manner with any of the other components of the pharmaceutical composition in which it is contained.

LMK235 may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release. In one or more preferred embodiments, the composition may be delivered subcutaneously.

Thus, LMK235 may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including, but not limited to, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the LMK235 into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of LMK235 administered can vary depending on various factors including, but not limited to, the weight, physical condition, and/or age of the subject, and/or the route of administration. Thus, the absolute weight of LMK235 included in a given unit dosage form can vary widely, and depends upon factors such as the species, age, weight, and physical condition of the subject, and/or the method of administration. Accordingly, it is not practical to set forth generally the amount that constitutes an amount of LMK235 effective for all possible applications. Those of ordinary skill in the art, however, can readily determine the appropriate amount with due consideration of such factors.

In one or more embodiments, the method can include administering sufficient LMK235 to provide a dose of, for example, from about 5 ng/kg to about 50 mg/kg to the subject although in one or more embodiments the methods may be performed by administering LMK235 in a dose outside this range. In some of these embodiments, the method includes administering sufficient LMK235 to provide a dose of from about 100 ng/kg to about 100 μg/kg to the subject, for example, a dose of from about 1 μg/kg to about 10 μg/kg. In one or more preferred embodiments, the method can include administering sufficient LMK235 to provide a dose of, for example, 5 μg/kg.

A single dose may be administered all at once, continuously for a prescribed period of time, or in multiple discrete administrations. When multiple administrations are used, the amount of each administration may be the same or different. For example, a dose of 5 μg per day may be administered as a single administration of 5 μg, continuously over 24 hours, as two or more equal administrations (e.g., two 2.5 μg administrations), or as two or more unequal administrations (e.g., a first administration of 4 μg followed by a second administration of 1 μg). When multiple administrations are used to deliver a single dose, the interval between administrations may be the same or different.

In one or more embodiments, LMK235 may be administered, for example, from a single dose to multiple doses per week, although in one or more embodiments the method can involve a course of treatment that includes administering doses of LMK235 at a frequency outside this range. When a course of treatment involves administering multiple doses within a certain period, the amount of each dose may be the same or different. For example, a course of treatment can include an initial loading dose, followed by a maintenance dose that is lower than the loading dose. Also, when multiple doses are used within a certain period, the interval between doses may be the same or be different.

In one or more embodiments, LMK235 may be administered from about once per month to multiple doses administered per day. In one or more preferred embodiments, LMK235 may be administered may administered daily.

In one or more preferred embodiments, LMK235 may be administered may administered daily for seven consecutive days. The two timepoints for initiating treatment after injury are merely exemplary. The data indicate that earlier treatment can alleviate pain better and return to naïve baseline more rapidly than if treatment is initiated at a time more distant from the time of injury. Treatment at the later timepoint in the model may be more representative of typical clinical circumstances. Thus, for long standing neuropathic pain the treatment may involve a higher dose, more frequent administration, or a longer duration of treatment.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the word “exemplary” means to serve as an illustrative example and should not be construed as preferred or advantageous over other embodiments.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXAMPLES

The present invention is illustrated by the following examples. It is to be understood that the particular examples, materials, amounts, and procedures are to be interpreted broadly in accordance with the scope and spirit of the invention as set forth herein.

### Animals, Species, Sex, and Numbers

BALBc/cAnNHsd and C57B1/6 male and female mice were purchased from Envigo (Indianapolis, IN) at 6-8 weeks old. The BALBc strain was chosen for most of the studies because of their gentle nature that allowed sensitivity testing on the whisker pad over an extended number of weeks. Animals were randomized to blinded treatment and control groups of n=4-11 as described for each study, giving 90% power to detect a treatment effect size of 65% compared to a baseline response of 5% at a significance level of 0.05.

Naïve, sham surgical, and surgically nerve injured animals all received equivalent isoflurane anesthesia. Animals were housed in the Animal Resources Center (ARC) housing facility maintained by the laboratory staff and Division of Laboratory and Animal Resources (DLAR) staff. All animals were kept on a reverse 12:12 light:dark cycle to assess all parameters during the animals' natural active time since rodents are naturally nocturnal animals. This reduced contribution of alterations of the circadian clock so that animals could be tested during their active time. Animals were monitored twice daily by DLAR staff and frequently more often by the laboratory staff. Animals were maintained on normal mouse breeder chow which is lower in soy protein content (known to alter hypersensitivity).

All animals were weighed once a week to insure maintenance of healthy weight gain. There were no group differences in weight throughout the 10-week study. Variation in weight and behavior was not evident among groups allowing blinded testing in these studies. The procedures for behavioral testing are standard methods in the field as approved by the American Pain Society and the International Association for the Study of Pain. The method of euthanasia is rapid and reliable and allows for dissection and collection of various tissues for further research. The method is consistent with recommendations of the Panel on Euthanasia of the American Veterinary Medical Association. Housing facilities are inspected and accredited by AAALAC.

### Trigeminal Inflammatory Compression (TIC) Nerve Injury Surgery

Mice underwent the TIC nerve injury surgery, a simplified non-ligation variant of the trigeminal CCI-ION model previously described (Lyons et al., Neuroscience 2015 Jun. 4; 295:126-138; Ma et al., MolBrain 2012 Dec. 28; 5:44). Briefly, mice were anesthetized with isoflurane (3-4%) and a piece of chromic gut suture (2 mm length, 6-0) was placed parallel to the infraorbital nerve (ION) adjacent to the maxillary bone infraorbital fissure at a site just under the lower eyelid. Naïve controls underwent anesthesia only. The chronic trigeminal inflammatory compression (TIC) nerve injury model induces measurable changes in evoked reflexive or higher order pain-like and anxiety-like behaviors but are not otherwise detectable.

### Foramen Rotundum Inflammatory Constriction Trigeminal Infraorbital Nerve (FRICT-ION) Injury Surgery

The FRICT-ION chronic neuropathic pain model combines the chemical irritation of chromic gut suture with some physical compression of the maxillary branch (V2) of the trigeminal nerve. FRICT-ION model was induced in 9-week-old mice anesthetized briefly with isoflurane (2-3%). Intraoral surgical incision was made with a small scalpel puncture at the buccal cheek crease. A piece of chromic gut suture (3 mm length, 4-0) was placed parallel to the infraorbital nerve (ION) and pushed into the tight space to follow the trigeminal nerve as it passes into the foramen rotundum of the skull. The model simulates blunt force traumatic injury to the nerve, residual wound debris, or other irritation.

### Drug Treatments

The HDAC4/5 inhibitor LMK235 (s.c., 5 mg/kg)(N-[[6-(Hydroxyamino)-6-oxohexyl]oxy]-3,5-dimethyl-benzamide) (Cayman Chemical Co., Ann Arbor, MI) was used at previously published dilutions (Bennett G J, Xie Y K. Pain 1988 April; 33:87-107). LMK235 is selective for HDAC 4 (IC₅₀=12 nM) and HDAC 5 (IC₅₀=4 nM) over other HDACs (IC₅₀s=56, 320, 850, 880, and 1,280 nM for HDACs 6, 1, 11, 2, and 8, respectively). All treatments with the HDACi (5 mg/kg) or vehicle were given daily for seven consecutive days in the neck scruff (s.c.) for comparisons to naïve control mice.

Post-Treatment in TIC Mice at Three Weeks with HDACi (n=11; n=4/Group Except Control)

In the first post-treatment study, male C57B1/6 mice were subcutaneously (s.c.) injected daily for seven days beginning in week 3 following the trigeminal nerve insult induced surgically. Withdrawal thresholds were tested weekly with von Frey fiber stimulation on the snout. One TIC mouse behaving erratically on day 1-7 was eliminated from the study.

Post-Treatment in TIC Mice with HDACi at 8 Weeks (n=12; n=4/Group)

In the second post-treatment study, C57B1/6 mice were treated daily for 14 days in week 8 and week 9 after the nerve insult. Withdrawal thresholds were determined weekly with von Frey fiber stimulation on the snout until experiment end.

Post-Treatment in FRICT-ION Mice at 3 Weeks with HDACi (n=56; n=8/Group)

On the day of FRICT-ION model induction in BALBc mice, LMK235 was administered and treatment was continued for seven days. Withdrawal thresholds were determined weekly with von Frey fiber stimulation on the snout. Cold sensitivity on the snout and anxiety were tested once prior to experiment end in week 8-9.

### Behavioral Assessments

Testing was done by an observer blinded to study group. Von Frey assessment of snout withdrawal threshold was conducted prior to and at least once a week after nerve injury to confirm development of craniofacial neuropathic pain. An additional assessment was performed on day 3 in the BALBc FRICT-ION mice to assess and compare the rapid reversal of hypersensitivity by LMK235. Separate comparisons were made to surgical sham and naïve animals that did not receive any surgery but had all behavioral tests, with all animals participating in anesthesia and weekly behavioral testing.

### Reflexive Mechanical and Cold Threshold Test

Mechanical and cold sensitivity was modified for face as previously described (Montera M A, Westlund K N, Bio-protocol2020 April 20; 10(8):e3591-e3591; Lyons et al., Neuroscience 2015 Jun. 4; 295:126-138; Ma et al., Mol Brain 2012 Dec. 28; 5:44; Montera et al., Channels (Austin). 2021 December; 15(1):31-37). Briefly, animals were gently restrained and the mechanical threshold for nocifensive head withdrawal was determined using calibrated von Frey filaments (0.008 g-6.0 g) using the Up-Down method (Chaplan et al., J Neurosci Methods 1994 July; 53(1):55-63; FIG. 2). Cold sensitivity was determined by applying a 10° C. cold coil to the ION's receptive field and measuring the time until an animal withdrew its head (Montera et al., Channels (Austin). 2021 December; 15(1):31-37).

### Higher Order Behavioral Tests

Anxiety-related behaviors were assessed in a manner typical in the literature—i.e., once at the end in week 8 since anxiety develops only in mice 6-8 weeks after persisting nerve injury (e.g., Lyons et al., Neuroscience 2015 Jun. 4; 295:126-138). The effects of HDACi on chronic craniofacial pain-related behaviors were assessed using light/dark place preference test and zero maze.

### Light/Dark Place Preference Test

Animals were exposed to acoustic startle (5 minutes) immediately prior to placement in the light/dark place preference box as in a previous study (Lyons et al., Neuroscience 2015 Jun. 4; 295:126-138). Mils acoustic startle stress just prior to the test induces panic anxiety-like behavior with increased time and entries in the open arms compared to controls (de Paula HMG, Hoshino K, Behav Brain Res 2003 Dec. 17; 147(1-2):157-162; de Paula HMG, Hoshino K, Physiol Behav 2004 January; 80(4):459-464; Kontinen et al., Pain 1999 March;80(1-2):341-346). In the light/dark box place preference task (total time 10 minutes) anxiety-like behaviors were recorded as: (i) total time spent in the light area, (ii) number of entries into the light area, (iii) number of rearing events/exploratory behavior, (iv) latency of the first transition into the light chamber, and (v) latency of first re-entry (transition) back into the dark chamber.

### Zero Maze

The zero maze task is widely used to test anxiety-like behavior (Kontinen et al., Pain 1999 March;80(1-2):341-346; Belzung C, Griebel G, Behav Brain Res 2001 Nov. 1; 125(1-2):141-149; Roeska et al., Pain 2008; 139(2):349-357. As previously reported, the zero maze consists of two open and two closed areas arranged around a zero-shape (Lyons et al., Neuroscience 2015 Jun. 4; 295:126-138). Behaviors were recorded for five minutes and analyzed off-line by an observer blinded to experimental group for: (i) time spent in open areas, (ii) number of transitions, and (iii) number of head dips into the open areas. Anxiety-like response to this perceived threat results in decreased time spent in the open areas and decreased entries into the maze's open areas (Belzung C, Griebel G, Behav Brain Res 2001 Nov. 1;125(1-2):141-149).

### Conditioned Place Preference (CPP) Test of Addictive Potential

Mice were assigned either vehicle, morphine (5 mg/kg) or LMK235 (5 mg/kg) and assigned cubicle designation for groups (n=3) as follows: Vehicle-Blank, Vehicle-Striped, Morphine-Blank, Morphine-Striped, LMK-Blank, LMK-Striped. For baseline collection, mice were placed in the middle of the box and allowed access to both chambers for 15 minutes. Time spent in each chamber was recorded. For the subsequent three test days, mice were injected with their assigned drug and placed in their assigned chamber, without access to the other chamber, for 30 minutes. On the final test day, mice were placed in the middle of the box and allowed access to both chambers for 15 minutes. Time spent in each chamber was recorded.

### Whole-Cell Patch-clamp Electrophysiology

Neurons with a diameter of <30 μm were identified by infrared differential interference contrast (IR-DIC) imaging with a microscope connected to an Olympus digital camera. Current clamp recordings were performed using a Molecular Devices Multiclamp 700B (Scientifica, UK). Signals were filtered at 5 KHz, acquired at 50 KHz using a Molecular Devices 1550B converter (Scientifica, UK) and recorded using Clampex 11 software (Molecular Devices, Scientifica, UK). Electrodes were pulled with a Zeitz puller (Werner Zeitz, Martinsreid, Germany) from borosilicate thick glass (GC150F, Sutter Instruments). Electrode resistance was 5-8 MΩ. Bridge balance was applied to all recordings. Intracellular solution contained (in mM) 125 K-gluconate, 6 KCl, 10 HEPES, 0.1 EGTA, 2 Mg-ATP, pH 7.3 with KOH, and osmolarity of 290-310 mOsm. Artificial cerebrospinal fluid (aCSF) contained (in mM) 113 NaCl, 3 KCl, 25 NaHCO₃, 1 NaH₂PO₄, 2 CaCl₂), 2 MgCl2, and 11-glucose. For whole-cell current clamp recordings, to evaluate the basic input-output action potential frequency response to hyperpolarization and depolarization, DC current was injected from 0 pA to +200 pA in 10-pA increments for a duration of 500 milliseconds at the cell's intrinsic resting membrane potential. Data acquisition was sampled at 20 kHz and filtered at 2.4 kHz. Recordings with a series resistance greater than 20 MΩ were discarded, and series resistance was compensated to 70%. In response to a −100 pA hyperpolarizing pulse with 500 millisecond duration, voltage sag amplitude was measured. Sag ratio was calculated using the following equation as described previously (Fan et al., Front Cell Neurosci 2016 Mar. 24; 10:74):

$$Sag ratio = [ ( {V}_{p e a k} - {V}_{s s} ) / {V}_{s s} ] \times 100 ,$$

where Vₚₑₐₖ is the maximum voltage deflection and Vₛₛ is the steady state voltage at the end of the hyperpolarizing pulse.

### RNA Profiling

Gene expression profiling was performed on TG from mice with/without nerve injury during the acute phase (three weeks post injury) in TIC mice and after transition to chronic pain (10 weeks post injury) in FRICT-ION mice by Quick Biology Inc. (Monrovia, CA).

### RNAseq

Total RNA from ipsilateral TG was isolated using the Rneasy Mini Kit (Qiagen, Valencia, CA). Gene expression profiling and analysis with RNA was done by Quick Biology Inc. (Monrovia, CA). RNAseq library preparation was performed by Quick Biology Inc. (Monrovia, CA), sequencing was performed using an Illumina HiSeq 4000 at 40 million reads per sample. The reads were first mapped to the latest UCSC transcript set using Bowtie2 version 2.1.0 and the gene expression level was estimated using RSEM v1.2.15. Differentially expressed genes were identified using the edgeR program. Genes showing altered expression with p<0.05 were considered differentially expressed.

Results are provided in Table 1, Table 2, and Table 3.

TABLE 1
RNAseq differentially expressed genes in TG of male FRICT-ION mice compared to naives

| Gene_symbol | Name | Fold change | P-Value |
|---|---|---|---|
| Rbm14-rbm4 | NA | 32.66 | 3.31 × 10⁻³ |
| Hoxc8 | homeobox C8 | 25.26 | 4.63 × 10 ⁻⁵ |
| Tmprss11a | transmembrane protease; serine 11a | 18.53 | 4.27 × 10 ⁻² |
| Ttr | transthyretin | 12.72 | 5.36 × 10 ⁻⁴ |
| Hoxb9 | homeobox B9 | 11.88 | 1.73 × 10 ⁻⁷ |
| Kcne2 | potassium voltage-gated channel; Isk-related | 11.10 | 1.24 × 10 ⁻² |
| | subfamily; gene 2 | | |
| Gh | growth hormone | 8.06 | 4.46 × 10 ⁻³ |
| Sostdc1 | sclerostin domain containing 1 | 7.93 | 1.03 × 10 ⁻² |
| Folr1 | folate receptor 1 (adult) | 6.65 | 3.28 × 10 ⁻² |
| Hoxd8 | homeobox D8 | 5.99 | 6.46 × 10 ⁻⁸ |
| Ngp | neutrophilic granule protein | 5.95 | 3.75 × 10 ⁻⁶ |
| Car12 | carbonic anyhydrase 12 | 5.36 | 1.94 × 10 ⁻³ |
| S100a9 | S100 calcium binding protein A9 (calgranulin B) | 5.30 | 5.98 × 10 ⁻⁷ |
| Cldn2 | claudin 2 | 5.12 | 4.35 × 10 ⁻² |
| Gvin1 | GTPase; very large interferon inducible 1 | 5.03 | 4.89 × 10 ⁻³ |
| Tac2 | tachykinin 2 | 4.84 | 8.60 × 10 ⁻8 |
| Hoxd9 | homeobox D9 | 4.83 | 5.80 × 10 ⁻⁴ |
| Mira | mistral long non-coding RNA | 4.40 | 3.02 × 10 ⁻⁶ |
| S100a8 | S100 calcium binding protein A8 (calgranulin A) | 4.27 | 2.34 × 10 ⁻⁵ |
| Nhlh1 | nescient helix loop helix 1 | 4.22 | 1.92 × 10 ⁻³ |
| Nmu | neuromedin U | 4.02 | 2.48 × 10 ⁻⁷ |
| Hoxa7 | homeobox A7 | 3.93 | 2.72 × 10 ⁻⁷ |
| Tmem235 | transmembrane protein 235 | 3.92 | 9.52 × 10 ⁻⁶ |
| Hoxa6 | homeobox A6 | 3.86 | 1.02 × 10 ⁻⁶ |
| Tex15 | testis expressed gene 15 | 3.84 | 4.08 × 10 ⁻⁷ |
| Hoxb8 | homeobox B8 | 3.83 | 8.89 × 10 ⁻⁸ |
| Ankfn1 | ankyrin-repeat and fibronectin type III domain | 3.75 | 4.36 × 10 ⁻⁵ |
| | containing 1 | | |
| 4833423E24Rik | RIKEN cDNA 4833423E24 gene | 3.72 | 3.69 × 10 ⁻⁶ |
| Hoxb7 | homeobox B7 | 3.72 | 1.90 × 10 ⁻⁸ |
| Ltf | lactotransferrin | 3.65 | 1.82 × 10 ⁻⁴ |
| Tmem72 | transmembrane protein 72 | 3.64 | 4.87 × 10 ⁻² |
| F2rl2 | coagulation factor II (thrombin) receptor-like 2 | 3.57 | 1.23 × 10 ⁻⁵ |
| Krt18 | keratin 18 | 3.53 | 3.78 × 10 ⁻² |
| Ackr4 | atypical chemokine receptor 4 | 3.51 | 1.25 × 10 ⁻² |
| Aplnr | apelin receptor | 3.39 | 4.31 × 10 ⁻⁵ |
| Otx2 | orthodenticle homolog 2 | 3.34 | 3.50 × 10 ⁻² |
| Kl | klotho | 3.18 | 3.57 × 10 ⁻² |
| Tgtp1 | T cell specific GTPase 1 | 3.13 | 1.33 × 10 ⁻³ |
| A730008H23Rik | RIKEN cDNA A730008H23 gene | 3.11 | 1.76 × 10 ⁻² |
| Cdh3 | cadherin 3 | 3.07 | 2.86 × 10 ⁻⁶ |
| Glp2r | glucagon-like peptide 2 receptor | 3.03 | 9.40 × 10 ⁻⁴ |
| Amd1 | S-adenosylmethionine decarboxylase 1 | 2.86 | 1.18 × 10 ⁻² |
| Robo3 | roundabout homolog 3 (Drosophila) | 2.84 | 2.28 × 10 ⁻⁴ |
| Arsj | arylsulfatase J | 2.83 | 1.30 × 10 ⁻³ |
| Gbx1 | gastrulation brain homeobox 1 | 2.78 | 9.27 × 10 ⁻⁶ |
| Hoxc6 | homeobox C6 | 2.77 | 8.73 × 10 ⁻⁷ |
| Ebf2 | early B cell factor 2 | 2.73 | 1.92 × 10 ⁻⁸ |
| Slfn4 | schlafen 4 | 2.72 | 2.25 × 10 ⁻² |
| Thbs1 | thrombospondin 1 | 2.70 | 1.16 × 10 ⁻⁷ |
| Camp | cathelicidin antimicrobial peptide | 2.69 | 6.16 × 10 ⁻³ |
| Gmnc | geminin coiled-coil domain containing | 2.64 | 2.03 × 10 ⁻² |
| Cldn1 | claudin 1 | 2.62 | 3.05 × 10 ⁻³ |
| Grp | gastrin releasing peptide | 2.52 | 1.21 × 10 ⁻⁵ |
| Lmx1b | LIM homeobox transcription factor 1 beta | 2.47 | 2.60 × 10 ⁻⁵ |
| Map3k15 | mitogen-activated protein kinase kinase kinase 15 | 2.45 | 4.29 × 10 ⁻³ |
| Adh1 | alcohol dehydrogenase 1 (class I) | 2.45 | 1.12 × 10 ⁻² |
| Nmur2 | neuromedin U receptor 2 | 2.44 | 6.64 × 10 ⁻⁵ |
| Serpinb1b | serine (or cysteine) peptidase inhibitor; clade B; | 2.43 | 7.72 × 10 ⁻¹⁰ |
| | member 1b | | |
| Hoxb6 | homeobox B6 | 2.41 | 1.22 × 10 ⁻⁷ |
| Lrrc10b | leucine rich repeat containing 10B | 2.41 | 3.69 × 10 ⁻⁴ |
| Pdyn | prodynorphin | 2.35 | 1.26 × 10 ⁻¹² |
| Skor2 | SKI family transcriptional corepressor 2 | 2.34 | 2.44 × 10 ⁻⁶ |
| C3 | complement component 3 | 2.34 | 1.40 × 10 ⁻⁶ |
| Slco4c1 | solute carrier organic anion transporter family; | 2.34 | 1.15 × 10 ⁻³ |
| | member 4C1 | | |
| Oxtr | oxytocin receptor | 2.31 | 1.01 × 10 ⁻⁷ |
| Gm16740 | predicted gene; 16740 | 2.26 | 4.48 × 10 ⁻² |
| AI848285 | NA | 2.22 | 2.36 × 10 ⁻⁷ |
| Bmp3 | bone morphogenetic protein 3 | 2.20 | 1.57 × 10 ⁻³ |
| Podn | podocan | 2.19 | 9.70 × 10 ⁻³ |
| Egr3 | early growth response 3 | 2.18 | 5.17 × 10 ⁻⁸ |
| Nrgn | neurogranin | 2.17 | 5.65 × 10 ⁻⁷ |
| Ebf1 | early B cell factor 1 | 2.17 | 1.91 × 10 ⁻¹⁰ |
| Cckbr | cholecystokinin B receptor | 2.15 | 8.82 × 10 ⁻³ |
| Il1a | interleukin 1 alpha | 2.15 | 7.17 × 10 ⁻³ |
| Ebf3 | early B cell factor 3 | 2.14 | 2.44 × 10 ⁻¹¹ |
| Satb2 | special AT-rich sequence binding protein 2 | 2.13 | 8.43 × 10 ⁻³ |
| P2rx4 | purinergic receptor P2X; ligand-gated ion channel 4 | 2.12 | 1.28 × 10 ⁻² |
| I120ra | interleukin 20 receptor; alpha | 2.12 | 6.20 × 103 |
| Cpxm2 | carboxypeptidase X 2 (M14 family) | 2.12 | 1.18 × 10 ⁻³ |
| Tex11 | testis expressed gene 11 | 2.12 | 9.47 × 10 ⁻³ |
| Ect2 | ect2 oncogene | 2.11 | 1.56 × 10 ⁻² |
| Hdac5 | histone deacetylase 5 | 2.10 | 2.14 × 10 ⁻² |
| Cd44 | CD44 antigen | 2.10 | 3.36 × 10 ⁻⁷ |
| Moxd1 | monooxygenase; DBH-like 1 | 2.09 | 2.17 × 10 ⁻² |
| Rab38 | RAB38; member RAS oncogene family | 2.09 | 4.93 × 10 ⁻³ |
| Igfbpl1 | insulin-like growth factor binding protein-like 1 | 2.08 | 3.47 × 10 ⁻² |
| Tlr6 | toll-like receptor 6 | 2.08 | 1.03 × 10 ⁻² |
| Mc5r | melanocortin 5 receptor | 2.08 | 1.42 × 10 ⁻² |
| Col23a1 | collagen; type XXIII; alpha 1 | 2.07 | 2.69 × 10 ⁻⁵ |
| Arhgap6 | Rho GTPase activating protein 6 | 2.07 | 2.34 × 10 ⁻⁷ |
| Tlx3 | T cell leukemia; homeobox 3 | 2.07 | 2.08 × 10 ⁻³ |
| Frzb | frizzled-related protein | 2.06 | 3.56 × 10 ⁻⁷ |
| Nts | neurotensin | 2.05 | 2.09 × 10 ⁻¹⁰ |
| 4932411E22Rik | RIKEN cDNA 4932411E22 gene | 2.04 | 9.23 × 10 ⁻⁴ |
| Myoc | myocilin | 2.04 | 5.30 × 10 ⁻⁴ |
| Sytl3 | synaptotagmin-like 3 | 2.02 | 3.09 × 10 ⁻² |
| Necab1 | N-terminal EF-hand calcium binding protein 1 | 2.02 | 2.39 × 10 ⁻⁸ |
| Lrriq3 | leucine-rich repeats and IQ motif containing 3 | 2.02 | 2.11 × 10 ⁻² |
| Camkv | CaM kinase-like vesicle-associated | 2.02 | 2.87 × 10 ⁻⁹ |
| E130309F12Rik | NA | 2.01 | 1.44 × 10 ⁻⁶ |
| Hdac9 | histone deacetylase 9 | 1.28 | 5.57 × 10 ⁻⁴ |
| Ache | acetylcholinesterase | −1.20 | 2.43 × 10 ⁻⁶ |
| Crhbp | corticotropin releasing hormone binding protein | −2.51 | 7.76 × 10 ⁻⁸ |
| Tph2 | tryptophan hydroxylase 2 | −4.51 | 1.27 × 10 ⁻⁷ |
| Slc6a4 | solute carrier family 6 (neurotransmitter transporter; | −4.86 | 4.23 × 10 ⁻⁶ |
| | serotonin); member 4 | | |
| C1ql4 | complement component 1; q subcomponent-like 4 | −5.44 | 7.65 × 10 ⁻⁶ |

TABLE 2
RNAseq differentially expressed genes in TG obtained from male FRICTION
mice treated with HDAC5 inhibitor, LMK235, compared to naive controls

| Gene_symbol | NAME | Fold change | P-Value |
|---|---|---|---|
| Rbm14-rbm4 | NA | 66.16 | 3.57 × 10⁻⁴ |
| Tmprss11a | transmembrane protease; serine 11a | 37.90 | 1.53 × 10⁻² |
| Ttr | transthyretin | 23.21 | 3.98 × 10⁻⁵ |
| Kcne2 | potassium voltage-gated channel; Isk-related | 17.29 | 4.00 × 10⁻³ |
| | subfamily; gene 2 | | |
| Sostdc1 | sclerostin domain containing 1 | 15.70 | 1.08 × 10⁻³ |
| Folr1 | folate receptor 1 (adult) | 11.92 | 6.97 × 10⁻³ |
| Cldn2 | claudin 2 | 8.46 | 1.00 × 10⁻² |
| Kcnj13 | potassium inwardly-rectifying channel; subfamily J; | 7.84 | 9.05 × 10⁻³ |
| | member 13 | | |
| Aqp1 | aquaporin 1 | 7.41 | 1.20 × 10⁻² |
| Krt18 | keratin 18 | 6.56 | 2.27 × 10⁻³ |
| Tmem72 | transmembrane protein 72 | 6.47 | 5.41 × 10⁻³ |
| Inmt | indolethylamine N-methyltransferase | 6.34 | 3.40 × 10⁻² |
| Car12 | carbonic anyhydrase 12 | 6.21 | 8.15 × 10⁻⁴ |
| F5 | coagulation factor V | 6.19 | 8.73 × 10⁻³ |
| Gvin1 | GTPase; very large interferon inducible 1 | 5.55 | 2.89 × 10⁻³ |
| Prr32 | proline rich 32 | 5.41 | 2.51 × 10⁻² |
| Otx2 | orthodenticle homolog 2 | 5.17 | 4.56 × 10⁻³ |
| Kl | klotho | 5.00 | 4.20 × 10⁻³ |
| Clic6 | chloride intracellular channel 6 | 4.92 | 1.28 × 10⁻² |
| Slc4a5 | solute carrier family 4; sodium bicarbonate | 4.83 | 1.74 × 10⁻² |
| | cotransporter; member 5 | | |
| 1500015O10Rik | RIKEN cDNA 1500015O10 gene | 4.68 | 4.05 × 10⁻² |
| Wfdc2 | WAP four-disulfide core domain 2 | 4.61 | 3.62 × 10⁻² |
| Mfrp | membrane-type frizzled-related protein | 4.30 | 3.56 × 10⁻² |
| Lbp | lipopolysaccharide binding protein | 3.97 | 2.45 × 10⁻² |
| Ackr4 | atypical chemokine receptor 4 | 3.74 | 8.58 × 10⁻³ |
| Fap | fibroblast activation protein | 3.16 | 1.34 × 10⁻² |
| Col8a2 | collagen; type VIII; alpha 2 | 3.06 | 2.24 × 10⁻² |
| Calml4 | calmodulin-like 4 | 2.88 | 3.34 × 10⁻² |
| Ccdc135 | NA | 2.84 | 3.57 × 10⁻² |
| Tgtp1 | T cell specific GTPase 1 | 2.49 | 1.06 × 10⁻² |
| Cldn1 | claudin 1 | 2.48 | 5.04 × 10⁻³ |
| Gmnc | geminin coiled-coil domain containing | 2.40 | 3.64 × 10⁻² |
| Igfbpl1 | insulin-like growth factor binding protein-like 1 | 2.39 | 9.72 × 10⁻³ |
| Crhr2 | corticotropin releasing hormone receptor 2 | 2.35 | 4.45 × 10⁻² |
| Postn | periostin; osteoblast specific factor | 2.24 | 2.29 × 10⁻² |
| Enpp2 | ectonucleotide pyrophosphatase/phosphodiesterase | 2.22 | 1.61 × 10⁻² |
| | 2 | | |
| Il1a | interleukin 1 alpha | 2.21 | 4.59 × 10⁻³ |
| Tnfsf13 | tumor necrosis factor (ligand) superfamily; member | 2.14 | 1.92 × 10⁻² |
| | 13 | | |
| 4833423E24Rik | RIKEN cDNA 4833423E24 gene | 2.10 | 9.74 × 10⁻³ |
| Lum | lumican | 2.08 | 1.04 × 10⁻² |
| Ache | acetylcholinesterase | −1.12 | 3.55 × 10⁻³ |
| Hdac5 | histone deacetylase 5 | −1.13 | 2.80 × 10⁻³ |

TABLE 3
RNAseq differentially expressed genes in TG obtained from male FRICT-ION mice treated
with HDAC5 inhibitor, LMK235, compared to FRICT-ION mice treated with Vehicle

| Gene_symbol | NAME | Fold change | P-Value |
|---|---|---|---|
| Gm8221 | predicted gene 8221 | 241.23 | 2.53 × 10⁻⁸ |
| Evi2a-evi2b | NA | 136.10 | 4.76 × 10⁻³ |
| Hist2h3c2 | histone cluster 2; H3c2 | 128.58 | 3.02 × 10⁻³ |
| Gm13306 | predicted gene 13306 | 48.23 | 3.47 × 10⁻⁵ |
| C1ql4 | complement component 1; q subcomponent-like 4 | 4.76 | 3.54 × 10⁻⁵ |
| Slc6a4 | solute carrier family 6 (neurotransmitter transporter; | 4.38 | 1.56 × 10⁻⁵ |
| | serotonin); member 4 | | |
| Tph2 | tryptophan hydroxylase 2 | 4.18 | 4.82 × 10⁻⁷ |
| Eif3j2 | eukaryotic translation initiation factor 3; subunit J2 | 3.88 | 6.31 × 10⁻³ |
| 1700047I17Rik2 | RIKEN cDNA 1700047117 gene 2 | 3.82 | 1.49 × 10⁻³ |
| Gata3 | GATA binding protein 3 | 3.74 | 8.44 × 10⁻⁸ |
| Gpx2 | glutathione peroxidase 2 | 3.17 | 2.41 × 10⁻³ |
| Tyrp1 | tyrosinase-related protein 1 | 3.03 | 2.55 × 10⁻⁴ |
| Cyp26b1 | cytochrome P450; family 26; subfamily b; polypeptide | 2.99 | 1.41 × 10⁻¹¹ |
| | 1 | | |
| Tmprss11d | transmembrane protease; serine 11d | 2.98 | 1.99 × 10⁻³ |
| lc6a2 | solute carrier family 6 (neurotransmitter transporter; | 2.91 | 3.19 × 10⁻⁴ |
| | noradrenalin); member 2 | | |
| Crhbp | corticotropin releasing hormone binding protein | 2.79 | 1.62 × 10⁻⁹ |
| Vmn2r84 | vomeronasal 2; receptor 84 | 2.71 | 6.79 × 10⁻³ |
| Ppp1r1c | protein phosphatase 1; regulatory (inhibitor) subunit 1C | 2.51 | 1.47 × 10⁻⁵ |
| Serpina3h | serine (or cysteine) peptidase inhibitor; clade A; | 2.51 | 2.85 × 10⁻⁵ |
| | member 3H | | |
| Irx4 | Iroquois related homeobox 4 (Drosophila) | 2.51 | 2.62 × 10⁻³ |
| Shox2 | short stature homeobox 2 | 2.49 | 3.71 × 10⁻⁶ |
| 8430408G22Rik | RIKEN cDNA 8430408G22 gene | 2.46 | 1.32 × 10⁻⁴ |
| Dmrt3 | doublesex and mab-3 related transcription factor 3 | 2.46 | 2.68 × 10⁻³ |
| Gm833 | predicted gene 833 | 2.45 | 9.83 × 10⁻³ |
| Pde4c | phosphodiesterase 4C; cAMP specific | 2.45 | 8.92 × 10⁻⁵ |
| Sp8 | trans-acting transcription factor 8 | 2.42 | 1.17 × 10⁻³ |
| Mgl2 | macrophage galactose N-acetyl-galactosamine specific | 2.39 | 2.74 × 10⁻³ |
| | lectin 2 | | |
| Fev | FEV (ETS oncogene family) | 2.35 | 7.99 × 10⁻⁴ |
| C1ql2 | complement component 1; q subcomponent-like 2 | 2.34 | 2.91 × 10⁻⁸ |
| Ccdc153 | coiled-coil domain containing 153 | 2.32 | 2.61 × 10⁻² |
| Isl1 | ISLI transcription factor; LIM/homeodomain | 2.30 | 9.21 × 10⁻⁴ |
| Onecut3 | one cut domain; family member 3 | 2.22 | 1.16 × 10⁻³ |
| Calcb | calcitonin-related polypeptide; beta | 2.17 | 5.95 × 10⁻¹⁰ |
| Pappa2 | pappalysin 2 | 2.16 | 3.08 × 10⁻⁵ |
| Kcnmb1 | potassium large conductance calcium-activated channel; | 2.15 | 1.80 × 10⁻² |
| | subfamily M; beta member 1 | | |
| Cytl1 | cytokine-like 1 | 2.14 | 3.76 × 10⁻² |
| Gucy2f | guanylate cyclase 2f | 2.13 | 9.20 × 10⁻³ |
| Cutal | cutA divalent cation tolerance homolog-like | 2.10 | 6.16 × 10⁻³ |
| Nkx6-3 | NK6 homeobox 3 | 2.10 | 1.95 × 10⁻² |
| H19 | H19; imprinted maternally expressed transcript | 2.05 | 1.71 × 10⁻² |
| Armc3 | armadillo repeat containing 3 | 2.04 | 2.97 × 10⁻² |
| Amy2a5 | amylase 2a5 | 2.03 | 3.60 × 10⁻² |
| Myom1 | myomesin 1 | 2.01 | 6.07 × 10⁻³ |
| Slc18a2 | solute carrier family 18 (vesicular monoamine); | 2.01 | 9.55 × 10⁻⁶ |
| | member 2 | | |
| Acta2 | actin; alpha 2; smooth muscle; aorta | 2.01 | 1.51 × 10⁻³ |
| Sec16b | SEC16 homolog B (S. cerevisiae) | 1.98 | 4.14 × 10⁻⁴ |
| Asb4 | ankyrin repeat and SOCS box-containing 4 | 1.53 | 1.94 × 10⁻³ |
| P2rx2 | purinergic receptor P2X; ligand-gated ion channel; 2 | −1.84 | 1.62 × 10⁻² |
| Hdac5 | histone deacetylase 5 | −2.03 | 4.95 × 10⁻² |
| P2rx4 | purinergic receptor P2X; ligand-gated ion channel 4 | −2.08 | 3.31 × 10⁻² |
| Slfn4 | schlafen 4 | −2.32 | 4.63 × 10⁻² |
| Cckbr | cholecystokinin B receptor | −2.32 | 6.63 × 10⁻⁴ |
| Hoxd8 | homeobox D8 | −4.14 | 1.02 × 10⁻⁵ |
| Hoxc8 | homeobox C8 | −8.22 | 2.32 × 10⁻³ |
| Hoxb9 | homeobox B9 | −16.50 | 7.21 × 10⁻⁹ |
| Gh | growth hormone | −25.22 | 9.19 × 10⁻⁵ |

### GO Analysis

GO analysis on the RNAseq data was conducted by Quick Biology Inc. (Monrovia, CA). Genes corresponding with wound healing, nerve regeneration and repair, pain, inflammation, and behavior with at least a two-fold change (po0.y5) were identified from the RNAseq gene expression profile. Gene ontologies were categorized as either biological process, cellular component, or molecular function. Results are provided in Table 4 and Table 5.

TABLE 4
Percentage of differentially expressed genes in TG from male FRICT-ION mice given daily
LMK235 or Vehicle, compared to naive mice and with each other from GO analysis

| | FRICT-ION mice | | LMK235-treated mice | | LMK235-treated mice | |
|---|---|---|---|---|---|---|
| GO Description | versus Naive | | versus Naive | | versus Vehicle | |
| Response to wounding | 8% | (33/406) | 11% | (14/131) | 8% | (18/235) |
| and wound healing | | | | | | |
| Inflammatory response | 7% | (28/406) | 9% | (12/131) | 1% | (2/235) |
| *Chronic inflammatory | *0.5% | (2/406) | *Chronic inflammatory | | 1% | (2/235) |
| response | | | response was not | | | |
| | | | presented in the data | | | |
| Negative regulation of | No data | | No data | | 2% | (5/235) |
| inflammatory response | | | | | | |
| Perception of pain and | 4% | (15/406) | 3% | (4/131) | 7% | (16/235) |
| response to pain | | | | | | |
| Locomotory and | 5% | (21/406) | *Behaviors were not | | 7% | (17/235) |
| chemosensory behavior | | | presented in the data | | | |
| Grooming, social, and | 2% | (9/406) | *Behaviors were not | | 3% | (7/235) |
| aggressive behavior | | | presented in the data | | | |
| Learning or memory | 4% | (18/406) | *Learning or memory | | 5% | (12/235) |
| | | | were not present | | | |
| | | | in the data | | | |
| Startle response | 1% | (3/406) | *Startle response was | | 1% | (2/235) |
| | | | not present in the data | | | |
| Negative regulation of | 3% | (11/406) | 6% | (8/131) | No data | |
| cytokine production | | | | | | |

TABLE 5

| | | | FRICT-ION + | FRICT-ION + |
|---|---|---|---|---|
| Largest Pain Related Changes | | FRICT-ION | LMK235 | LMK235 |
| from the GO Analysis | | VS | VS | VS |
| (not included in the Heat Maps) | Gene | Naive | Naive | FRICT-ION |
| Transcription/Regulation of gene | Hoxb9 | 11.88 | | −16.5 |
| expression | Hoxc8 | 25.26 | | −8.22 |
| | Hoxd8 | 5.99 | | −4.14 |
| | Otx2 | 3.34 | 5.17 | |
| Pain | Aqp1 | | 7.4 | |
| Neurogenesis/Neuron | Folr1 | 6.65 | 11.91 | |
| differentiation/ | | | | |
| Axongenesis/Axon | | | | |
| regeneration/Cell division | | | | |
| Ion channel/Ion binding/Ion | Kcne2 | 11.1 | 17.29 | |
| transport | | | | |
| | Kcnj13 | | 7.84 | |
| Immune/Inflammatory/Cytokine/ | Gh | 8.06 | | −25.22 |
| Hormone/Neurotransmitter | Gm13306 | −25.18 | | 48.23 |
| | S100a9 | 5.3 | | −2.74 |
| | Ttr | 12.72 | 23.21 | |

TABLE 6

| Mouse Genes Up-Regulated | | | | | | |
|---|---|---|---|---|---|---|
| and Down-Regulated | | FRICT-ION | | | LMK235-Treated | |
| Also Reported in | | Vs | | | FRICT-ION Vs | |
| Human Trigeminal Neuralgia | | Naive | | | Naive | |
| Gene | | Fold- | | | Fold- | |
| Symbol | NAME | change | P-Value | FDR | change | P-Value |
| Arfgef2 | ADP-ribosylation factor | 1.01 | 8.02 × 10⁻¹ | 9.38 × 10⁻¹ | 1.02 | 6.82 × 10⁻¹ |
| | guanine nucleotide- | | | | | |
| | exchange factor 2 (brefeldin | | | | | |
| | A-inhibited) | | | | | |
| Astn2 | astrotactin 2 | −1.02 | 7.72 × 10⁻¹ | 9.26 × 10⁻¹ | 1.04 | 5.61 × 10⁻¹ |
| Bmp2 | bone morphogenetic protein | 1.13 | 3.27 × 10⁻¹ | 6.66 × 10⁻¹ | 1.11 | 4.04 × 10⁻¹ |
| | 2 | | | | | |
| Bmp3 | bone morphogenetic protein | 2.20 | 1.57 × 10⁻³ | 2.81 × 10⁻² | −1.23 | 4.34 × 10⁻¹ |
| | 3 | | | | | |
| Bmp4 | bone morphogenetic protein | 1.13 | 5.55 × 10⁻¹ | 8.28 × 10⁻¹ | 1.18 | 4.15 × 10⁻¹ |
| | 4 | | | | | |
| Bmp5 | bone morphogenetic protein | 1.87 | 2.02 × 10⁻² | 1.54 × 10⁻¹ | 1.85 | 2.16 × 10⁻² |
| | 5 | | | | | |
| Cacna1a | calcium channel; voltage- | 1.10 | 6.89 × 10⁻² | 3.15 × 10⁻¹ | −1.06 | 2.67 × 10⁻¹ |
| | dependent; P/Q type; alpha | | | | | |
| | 1A subunit | | | | | |
| Cacna1d | calcium channel; voltage- | 1.01 | 8.68 × 10⁻¹ | 9.59 × 10⁻¹ | −1.03 | 7.06 × 10⁻¹ |
| | dependent; L type; alpha 1D | | | | | |
| | subunit | | | | | |
| Cacna1g | calcium channel; voltage- | 1.42 | 3.73 × 10⁻⁴ | 9.63 × 10⁻³ | −1.00 | 9.71 × 10⁻¹ |
| | dependent; T type; alpha 1G | | | | | |
| | subunit | | | | | |
| Cacna1h | calcium channel; voltage- | 1.34 | 1.91 × 10⁻³ | 3.17 × 10⁻² | −1.11 | 2.69 × 10⁻¹ |
| | dependent; T type; alpha 1H | | | | | |
| | subunit | | | | | |
| Cacna1i | calcium channel; voltage- | 1.21 | 6.55 × 10⁻³ | 7.32 × 10⁻² | −1.14 | 6.20 × 10⁻² |
| | dependent; alpha 1I subunit | | | | | |
| Cacnb1 | calcium channel; voltage- | 1.11 | 3.79 × 10⁻² | 2.24 × 10⁻¹ | −1.02 | 6.74 × 10⁻¹ |
| | dependent; beta 1 subunit | | | | | |
| Clcn1 | chloride channel 1 | −1.27 | 2.53 × 10⁻¹ | 5.99 × 10⁻¹ | −1.33 | 1.67 × 10⁻¹ |
| Clcn2 | chloride channel 2 | −1.12 | 8.61 × 10⁻² | 3.54 × 10⁻¹ | −1.11 | 1.11 × 10⁻¹ |
| Clic5 | chloride intracellular | −1.03 | 7.54 × 10⁻¹ | 9.18 × 10⁻¹ | −1.08 | 4.43 × 10⁻¹ |
| | channel 5 | | | | | |
| Eef2 | eukaryotic translation | −1.06 | 3.53 × 10⁻¹ | 6.89 × 10⁻¹ | −1.05 | 4.62 × 10⁻¹ |
| | elongation factor 2 | | | | | |
| Gabra5 | gamma-aminobutyric acid | 1.26 | 1.75 × 10⁻⁴ | 5.70 × 10⁻³ | 1.02 | 7.71 × 10⁻¹ |
| | (GABA) A receptor; subunit | | | | | |
| | alpha 5 | | | | | |
| Gabra6 | gamma-aminobutyric acid | −1.77 | 2.14 × 10⁻¹ | 5.55 × 10⁻¹ | 1.18 | 7.11 × 10⁻¹ |
| | (GABA) A receptor; subunit | | | | | |
| | alpha 6 | | | | | |
| Gabre | gamma-aminobutyric acid | −1.33 | 1.16 × 10⁻¹ | 4.12 × 10⁻¹ | −1.22 | 2.73 × 10⁻¹ |
| | (GABA) A receptor; subunit | | | | | |
| | epsilon | | | | | |
| Gabrg1 | gamma-aminobutyric acid | 1.11 | 2.41 × 10⁻¹ | 5.86 × 10⁻¹ | 1.26 | 7.39 × 10⁻³ |
| | (GABA) A receptor; subunit | | | | | |
| | gamma 1 | | | | | |
| Jakmip1 | janus kinase and | 1.12 | 3.13 × 10⁻² | 2.00 × 10⁻¹ | 1.02 | 7.51 × 10⁻¹ |
| | microtubule interacting | | | | | |
| | protein 1 | | | | | |
| Kcna5 | potassium voltage-gated | 1.33 | 4.39 × 10⁻² | 2.42 × 10⁻¹ | −1.06 | 6.70 × 10⁻¹ |
| | channel; shaker-related | | | | | |
| | subfamily; member 5 | | | | | |
| Kcnc3 | potassium voltage gated | −1.01 | 7.82 × 10⁻¹ | 9.31 × 10⁻¹ | −1.05 | 3.14 × 10⁻¹ |
| | channel; Shaw-related | | | | | |
| | subfamily; member 3 | | | | | |
| Kcnd2 | potassium voltage-gated | 1.09 | 8.36 × 10⁻² | 3.47 × 10⁻¹ | 1.09 | 9.02 × 10⁻² |
| | channel; Shal-related family; | | | | | |
| | member 2 | | | | | |
| Kcnh2 | potassium voltage-gated | −1.01 | 7.51 × 10⁻¹ | 9.17 × 10⁻¹ | −1.06 | 1.81 × 10⁻¹ |
| | channel; subfamily H (eag- | | | | | |
| | related); member 2 | | | | | |
| Kcnh7 | potassium voltage-gated | 1.23 | 1.87 × 10⁻¹ | 5.21 × 10⁻¹ | 1.03 | 8.65 × 10⁻¹ |
| | channel; subfamily H (eag- | | | | | |
| | related); member 7 | | | | | |
| Kcnj6 | potassium inwardly- | 1.26 | 1.19 × 10⁻¹ | 4.18 × 10⁻¹ | −1.10 | 5.37 × 10⁻¹ |
| | rectifying channel; | | | | | |
| | subfamily J; member 6 | | | | | |
| Kcnk1 | potassium channel; | −1.07 | 1.67 × 10⁻¹ | 4.93 × 10⁻¹ | −1.02 | 7.50 × 10⁻¹ |
| | subfamily K; member 1 | | | | | |
| Kcns2 | K+ voltage-gated channel; | 1.40 | 5.66 × 10⁻³ | 6.63 × 10⁻² | 1.12 | 3.62 × 10⁻¹ |
| | subfamily S; 2 | | | | | |
| Kcnv1 | potassium channel; | 1.16 | 4.56 × 10⁻¹ | 7.64 × 10⁻¹ | 1.26 | 2.23 × 10⁻¹ |
| | subfamily V; member 1 | | | | | |
| Kif1b | kinesin family member 1B | −1.00 | 9.89 × 10⁻¹ | 9.97 × 10⁻¹ | 1.01 | 8.81 × 10⁻¹ |
| Maoa | monoamine oxidase A | −1.06 | 1.96 × 10⁻¹ | 5.33 × 10⁻¹ | 1.08 | 9.03 × 10⁻² |
| Mapk3 | mitogen-activated protein | −1.02 | 6.05 × 10⁻¹ | 8.52 × 10⁻¹ | −1.02 | 5.92 × 10⁻¹ |
| | kinase 3 | | | | | |
| Mpz | myelin protein zero | 1.13 | 8.73 × 10⁻¹ | 9.61 × 10⁻¹ | 1.07 | 9.25 × 10⁻¹ |
| Nacc2 | nucleus accumbens | −1.05 | 2.73 × 10⁻¹ | 6.19 × 10⁻¹ | −1.05 | 2.68 × 10⁻¹ |
| | associated 2; BEN and BTB | | | | | |
| | (POZ) domain containing | | | | | |
| Nav3 | neuron navigator 3 | 1.33 | 5.07 × 10⁻⁴ | 1.21 × 10⁻² | 1.12 | 1.87 × 10⁻¹ |
| Ncald | neurocalcin delta | 1.01 | 6.84 × 10⁻¹ | 8.89 × 10⁻¹ | 1.05 | 1.74 × 10⁻¹ |
| Ndnf | neuron-derived neurotrophic | 1.24 | 2.98 × 10⁻² | 1.95 × 10⁻¹ | 1.31 | 5.67 × 10⁻³ |
| | factor | | | | | |
| Net1 | neuroepithelial cell | −1.24 | 9.15 × 10⁻³ | 9.20 × 10⁻² | −1.11 | 2.12 × 10⁻¹ |
| | transforming gene 1 | | | | | |
| Neurod4 | neurogenic differentiation 4 | −1.78 | 6.79 × 10⁻² | 3.13 × 10⁻¹ | −1.13 | 6.76 × 10⁻¹ |
| Nf2 | neurofibromatosis 2 | −1.06 | 5.82 × 10⁻² | 2.87 × 10⁻¹ | −1.02 | 4.86 × 10⁻¹ |
| Nfasc | neurofascin | −1.07 | 1.40 × 10⁻¹ | 4.55 × 10⁻¹ | −1.09 | 6.15 × 10⁻² |
| Ngef | neuronal guanine nucleotide | 1.26 | 9.49 × 10⁻⁵ | 3.66 × 10⁻³ | −1.06 | 3.13 × 10⁻¹ |
| | exchange factor | | | | | |
| Nkain2 | Na+/K+ transporting | 1.20 | 8.88 × 10⁻³ | 9.05 × 10⁻² | 1.05 | 4.84 × 10⁻¹ |
| | ATPase interacting 2 | | | | | |
| Nova1 | neuro-oncological ventral | −1.00 | 9.95 × 10⁻¹ | 9.99 × 10⁻¹ | 1.04 | 3.05 × 10⁻¹ |
| | antigen 1 | | | | | |
| Nras | neuroblastoma ras oncogene | 1.03 | 5.55 × 10⁻¹ | 8.28 × 10⁻¹ | −1.00 | 9.66 × 10⁻¹ |
| Nrep | neuronal regeneration | 1.03 | 4.81 × 10⁻¹ | 7.83 × 10⁻¹ | 1.04 | 2.79 × 10⁻¹ |
| | related protein | | | | | |
| Nrg2 | neuregulin 2 | −1.06 | 6.54 × 10⁻¹ | 8.74 × 10⁻¹ | 1.04 | 7.24 × 10⁻¹ |
| Nrg3 | neuregulin 3 | 1.09 | 1.55 × 10⁻¹ | 4.76 × 10⁻¹ | 1.09 | 1.60 × 10⁻¹ |
| Nrp2 | neuropilin 2 | 1.00 | 9.64 × 10⁻¹ | 9.90 × 10⁻¹ | 1.07 | 3.21 × 10⁻¹ |
| Ntrk1 | neurotrophic tyrosine | −1.95 | 1.89 × 10⁻⁴ | 6.02 × 10⁻³ | −1.09 | 6.17 × 10⁻¹ |
| | kinase; receptor; type 1 | | | | | |
| Nts | neurotensin | 2.05 | 2.09 × 10⁻¹⁰ | 1.29 × 10⁻⁷ | 1.18 | 1.60 × 10⁻¹ |
| Plcl1 | phospholipase C-like 1 | −1.00 | 9.54 × 10⁻¹ | 9.87 × 10⁻¹ | 1.12 | 4.57 × 10⁻² |
| Scn2a1 | sodium channel; voltage- | 1.15 | 2.10 × 10⁻³ | 3.37 × 10⁻² | 1.10 | 2.62 × 10⁻² |
| | gated; type II; alpha 1 | | | | | |
| Scn3a | sodium channel; voltage- | 1.12 | 1.51 × 10⁻¹ | 4.71 × 10⁻¹ | 1.11 | 1.82 × 10⁻¹ |
| | gated; type III; alpha | | | | | |
| Scn5a | sodium channel; voltage- | 1.2 | 2.78 × 10⁻¹ | 6.24 × 10⁻¹ | 1.23 | 2.59 × 10⁻¹ |
| | gated; type V; alpha | | | | | |
| Scn7a | sodium channel; voltage- | 1.15 | 3.32 × 10⁻¹ | 6.71 × 10⁻¹ | 1.07 | 6.19 × 10⁻¹ |
| | gated; type VII; alpha | | | | | |
| Scn8a | sodium channel; voltage- | −1.12 | 2.49 × 10⁻³ | 3.80 × 10⁻² | −1.07 | 8.10 × 10⁻² |
| | gated; type VIII; alpha | | | | | |
| Scn9a | sodium channel; voltage- | −1.28 | 7.02 × 10⁻⁴ | 1.54 × 10⁻² | 1.01 | 8.54 × 10⁻¹ |
| | gated; type IX; alpha | | | | | |
| Slc6a4 | solute carrier family 6 | −4.86 | 4.23 × 10⁻⁶ | 3.07 × 10⁻⁴ | −1.11 | 7.48 × 10⁻¹ |
| | (neurotransmitter | | | | | |
| | transporter; serotonin); | | | | | |
| | member 4 | | | | | |
| Trak1 | trafficking protein; kinesin | −1.04 | 2.17 × 10⁻¹ | 5.57 × 10⁻¹ | −1.05 | 1.11 × 10⁻¹ |
| | binding 1 | | | | | |
| Trpc6 | transient receptor potential | −1.17 | 3.27 × 10⁻¹ | 6.66 × 10⁻¹ | −1.08 | 6.16 × 10⁻¹ |
| | cation channel; subfamily C; | | | | | |
| | member 6 | | | | | |
| Trpm2 | transient receptor potential | −1.08 | 1.00 × 10⁻¹ | 3.83 × 10⁻¹ | −1.10 | 3.87 × 10⁻² |
| | cation channel; subfamily | | | | | |
| | M; member 2 | | | | | |
| Trpm3 | transient receptor potential | −1.07 | 4.08 × 10⁻¹ | 7.33 × 10⁻¹ | 1.12 | 1.57 × 10⁻¹ |
| | cation channel; subfamily | | | | | |
| | M; member 3 | | | | | |
| Trpm4 | transient receptor potential | −1.19 | 1.12 × 10⁻¹ | 4.06 × 10⁻¹ | −1.14 | 2.32 × 10⁻¹ |
| | cation channel; subfamily | | | | | |
| | M; member 4 | | | | | |
| Trpm7 | transient receptor potential | 1.04 | 3.95 × 10⁻¹ | 7.22 × 10⁻¹ | 1.10 | 5.15 × 10⁻² |
| | cation channel; subfamily | | | | | |
| | M; member 7 | | | | | |
| Trps1 | trichorhinophalangeal | 1.11 | 1.13 × 10⁻¹ | 4.06 × 10⁻¹ | 1.14 | 3.61 × 10⁻² |
| | syndrome I (human) | | | | | |
| Trpv4 | transient receptor potential | 1.76 | 3.27 × 10⁻¹ | 6.66 × 10⁻¹ | 2.12 | 1.91 × 10⁻¹ |
| | cation channel; subfamily V; | | | | | |
| | member 4 | | | | | |
| Trpv6 | transient receptor potential | 1.10 | 6.11 × 10⁻¹ | 8.54 × 10⁻¹ | −1.07 | 7.19 × 10⁻¹ |
| | cation channel; subfamily V; | | | | | |
| | member 6 | | | | | |
| Unc80 | unc-80 homolog (C. | −1.03 | 5.86 × 10⁻¹ | 8.44 × 10⁻¹ | 1.07 | 1.84 × 10⁻¹ |
| | elegans) | | | | | |

| Mouse Genes Up-Regulated | | | | | |
|---|---|---|---|---|---|
| and Down-Regulated | | | LMK235-Treated | | |
| Also Reported in | | LMK235-Treated | FRICT-ION Vs | | |
| Human Trigeminal Neuralgia | | FRICT-ION Vs | Untreated FRICT-ION | | |
| Gene | | Naive | Fold- | | |
| Symbol | NAME | FDR | change | P-Value | FDR |
| Arfgef2 | ADP-ribosylation factor | 9.30 × 10⁻¹ | 1.01 | 8.74 × 10⁻¹ | 9.65 × 10⁻¹ |
| | guanine nucleotide- | | | | |
| | exchange factor 2 (brefeldin | | | | |
| | A-inhibited) | | | | |
| Astn2 | astrotactin 2 | 8.87 × 10⁻¹ | 1.0 | 3.80 × 10⁻¹ | 7.47 × 10⁻¹ |
| Bmp2 | bone morphogenetic protein | 8.21 × 10⁻¹ | −1.02 | 8.75 × 10⁻¹ | 9.65 × 10⁻¹ |
| | 2 | | | | |
| Bmp3 | bone morphogenetic protein | 8.35 × 10⁻¹ | −2.70 | 7.00 × 10−5 | 4.66 × 10⁻³ |
| | 3 | | | | |
| Bmp4 | bone morphogenetic protein | 8.27 × 10⁻¹ | 1.05 | 8.23 × 10⁻¹ | 9.50 × 10⁻¹ |
| | 4 | | | | |
| Bmp5 | bone morphogenetic protein | 4.22 × 10⁻¹ | −1.01 | 9.64 × 10⁻¹ | 9.89 × 10⁻¹ |
| | 5 | | | | |
| Cacna1a | calcium channel; voltage- | 7.47 × 10⁻¹ | −1.16 | 3.24 × 10⁻³ | 6.64 × 10⁻² |
| | dependent; P/Q type; alpha | | | | |
| | 1A subunit | | | | |
| Cacna1d | calcium channel; voltage- | 9.36 × 10⁻¹ | −1.04 | 5.84 × 10⁻¹ | 8.58 × 10⁻¹ |
| | dependent; L type; alpha 1D | | | | |
| | subunit | | | | |
| Cacna1g | calcium channel; voltage- | 9.95 × 10⁻¹ | −1.43 | 3.12 × 10⁻⁴ | 1.38 × 10⁻² |
| | dependent; T type; alpha 1G | | | | |
| | subunit | | | | |
| Cacna1h | calcium channel; voltage- | 7.48 × 10⁻¹ | −1.49 | 2.21 × 10⁻⁵ | 2.07 × 10⁻³ |
| | dependent; T type; alpha 1H | | | | |
| | subunit | | | | |
| Cacna1i | calcium channel; voltage- | 5.50 × 10⁻¹ | −1.39 | 4.03 × 10⁻⁶ | 6.44 × 10⁻⁴ |
| | dependent; alpha 1I subunit | | | | |
| Cacnb1 | calcium channel; voltage- | 9.28 × 10⁻¹ | −1.13 | 1.13 × 10⁻² | 1.41 × 10⁻¹ |
| | dependent; beta 1 subunit | | | | |
| Clcn1 | chloride channel 1 | 6.68 × 10⁻¹ | −1.05 | 8.20 × 10⁻¹ | 9.49 × 10⁻¹ |
| Clcn2 | chloride channel 2 | 6.26 × 10⁻¹ | 1.01 | 8.92 × 10⁻¹ | 9.69 × 10⁻¹ |
| Clic5 | chloride intracellular | 8.40 × 10⁻¹ | −1.05 | 6.50 × 10⁻¹ | 8.86 × 10⁻¹ |
| | channel 5 | | | | |
| Eef2 | eukaryotic translation | 8.50 × 10⁻¹ | 1.01 | 8.47 × 10⁻¹ | 9.58 × 10⁻¹ |
| | elongation factor 2 | | | | |
| Gabra5 | gamma-aminobutyric acid | 9.53 × 10⁻¹ | −1.23 | 4.81 × 10⁻⁴ | 1.88 × 10⁻² |
| | (GABA) A receptor; subunit | | | | |
| | alpha 5 | | | | |
| Gabra6 | gamma-aminobutyric acid | 9.38 × 10⁻¹ | 2.08 | 1.08 × 10⁻¹ | 4.53 × 10⁻¹ |
| | (GABA) A receptor; subunit | | | | |
| | alpha 6 | | | | |
| Gabre | gamma-aminobutyric acid | 7.50 × 10⁻¹ | 1.10 | 6.13 × 10⁻¹ | 8.71 × 10⁻¹ |
| | (GABA) A receptor; subunit | | | | |
| | epsilon | | | | |
| Gabrg1 | gamma-aminobutyric acid | 3.41 × 10⁻¹ | 1.14 | 1.31 × 10⁻¹ | 4.93 × 10⁻¹ |
| | (GABA) A receptor; subunit | | | | |
| | gamma 1 | | | | |
| Jakmip1 | janus kinase and | 9.47 × 10⁻¹ | −1.10 | 6.08 × 10⁻² | 3.44 × 10⁻¹ |
| | microtubule interacting | | | | |
| | protein 1 | | | | |
| Kcna5 | potassium voltage-gated | 9.27 × 10⁻¹ | −1.41 | 1.33 × 10⁻² | 1.56 × 10⁻¹ |
| | channel; shaker-related | | | | |
| | subfamily; member 5 | | | | |
| Kcnc3 | potassium voltage gated | 7.75 × 10⁻¹ | −1.04 | 4.65 × 10⁻¹ | 8.01 × 10⁻¹ |
| | channel; Shaw-related | | | | |
| | subfamily; member 3 | | | | |
| Kcnd2 | potassium voltage-gated | 5.93 × 10⁻¹ | −1.00 | 9.66 × 10⁻¹ | 9.90 × 10⁻¹ |
| | channel; Shal-related family; | | | | |
| | member 2 | | | | |
| Kcnh2 | potassium voltage-gated | 6.81 × 10⁻¹ | −1.04 | 3.06 × 10⁻¹ | 6.85 × 10⁻¹ |
| | channel; subfamily H (eag- | | | | |
| | related); member 2 | | | | |
| Kcnh7 | potassium voltage-gated | 9.73 × 10⁻¹ | −1.19 | 2.36 × 10⁻¹ | 6.29 × 10⁻¹ |
| | channel; subfamily H (eag- | | | | |
| | related); member 7 | | | | |
| Kcnj6 | potassium inwardly- | 8.80 × 10⁻¹ | −1.38 | 2.75 × 10⁻² | 2.33 × 10⁻¹ |
| | rectifying channel; | | | | |
| | subfamily J; member 6 | | | | |
| Kcnk1 | potassium channel; | 9.47 × 10⁻¹ | 1.05 | 2.82 × 10⁻¹ | 6.67 × 10⁻¹ |
| | subfamily K; member 1 | | | | |
| Kcns2 | K+ voltage-gated channel; | 7.99 × 10⁻¹ | −1.25 | 5.91 × 10⁻² | 3.40 × 10⁻¹ |
| | subfamily S; 2 | | | | |
| Kcnv1 | potassium channel; | 7.14 × 10⁻¹ | 1.09 | 6.37 × 10⁻¹ | 8.81 × 10⁻¹ |
| | subfamily V; member 1 | | | | |
| Kif1b | kinesin family member 1B | 9.76 × 10⁻¹ | 1.01 | 8.69 × 10⁻¹ | 9.64 × 10⁻¹ |
| Maoa | monoamine oxidase A | 5.93 × 10⁻¹ | 1.14 | 2.64 × 10⁻³ | 5.76 × 10⁻² |
| Mapk3 | mitogen-activated protein | 9.00 × 10⁻¹ | −1.00 | 9.87 × 10⁻¹ | 9.96 × 10⁻¹ |
| | kinase 3 | | | | |
| Mpz | myelin protein zero | 9.85 × 10⁻¹ | −1.05 | 9.47 × 10⁻¹ | 9.85 × 10⁻¹ |
| Nacc2 | nucleus accumbens | 7.48 × 10⁻¹ | −1.00 | 9.92 × 10⁻¹ | 9.97 × 10⁻¹ |
| | associated 2; BEN and BTB | | | | |
| | (POZ) domain containing | | | | |
| Nav3 | neuron navigator 3 | 6.85 × 10⁻¹ | −1.19 | 2.68 × 10⁻² | 2.29 × 10⁻¹ |
| Ncald | neurocalcin delta | 6.75 × 10⁻¹ | 1.03 | 3.41 × 10⁻¹ | 7.16 × 10⁻¹ |
| Ndnf | neuron-derived neurotrophic | 3.29 × 10⁻¹ | 1.06 | 5.55 × 10⁻¹ | 8.48 × 10⁻¹ |
| | factor | | | | |
| Net1 | neuroepithelial cell | 7.07 × 10⁻¹ | 1.12 | 1.68 × 10⁻¹ | 5.46 × 10⁻¹ |
| | transforming gene 1 | | | | |
| Neurod4 | neurogenic differentiation 4 | 9.29 × 10⁻¹ | 1.57 | 1.47 × 10⁻¹ | 5.16 × 10⁻¹ |
| Nf2 | neurofibromatosis 2 | 8.60 × 10⁻¹ | 1.04 | 2.22 × 10⁻¹ | 6.12 × 10⁻¹ |
| Nfasc | neurofascin | 5.50 × 10⁻¹ | −1.02 | 6.94 × 10⁻¹ | 9.06 × 10⁻¹ |
| Ngef | neuronal guanine nucleotide | 7.75 × 10⁻¹ | −1.34 | 7.33 × 10⁻⁷ | 1.68 × 10⁻⁴ |
| | exchange factor | | | | |
| Nkain2 | Na+/K+ transporting | 8.59 × 10⁻¹ | −1.14 | 5.34 × 10⁻² | 3.22 × 10⁻¹ |
| | ATPase interacting 2 | | | | |
| Nova1 | neuro-oncological ventral | 7.68 × 10⁻¹ | 1.04 | 3.01 × 10⁻¹ | 6.81 × 10⁻¹ |
| | antigen 1 | | | | |
| Nras | neuroblastoma ras oncogene | 9.94 × 10⁻¹ | −1.03 | 5.21 × 10⁻¹ | 8.31 × 10⁻¹ |
| Nrep | neuronal regeneration | 7.54 × 10⁻¹ | 1.01 | 7.07 × 10⁻¹ | 9.11 × 10⁻¹ |
| | related protein | | | | |
| Nrg2 | neuregulin 2 | 9.41 × 10⁻¹ | 1.10 | 4.16 × 10⁻¹ | 7.71 × 10⁻¹ |
| Nrg3 | neuregulin 3 | 6.66 × 10⁻¹ | −1.00 | 9.77 × 10⁻¹ | 9.93 × 10⁻¹ |
| Nrp2 | neuropilin 2 | 7.79 × 10⁻¹ | 1.07 | 3.41 × 10⁻¹ | 7.16 × 10⁻¹ |
| Ntrk1 | neurotrophic tyrosine | 9.10 × 10⁻¹ | 1.80 | 9.90 × 10⁻⁴ | 3.13 × 10⁻² |
| | kinase; receptor; type 1 | | | | |
| Nts | neurotensin | 6.66 × 10⁻¹ | −1.74 | 4.23 × 10⁻⁷ | 1.16 × 10⁻⁴ |
| Plcl1 | phospholipase C-like 1 | 5.13 × 10⁻¹ | 1.13 | 3.94 × 10⁻² | 2.78 × 10⁻¹ |
| Scn2a1 | sodium channel; voltage- | 4.41 × 10⁻¹ | −1.04 | 3.85 × 10⁻¹ | 7.51 × 10⁻¹ |
| | gated; type II; alpha 1 | | | | |
| Scn3a | sodium channel; voltage- | 6.82 × 10⁻¹ | −1.01 | 9.15 × 10⁻¹ | 9.76 × 10⁻¹ |
| | gated; type III; alpha | | | | |
| Scn5a | sodium channel; voltage- | 7.41 × 10⁻¹ | 1.01 | 9.76 × 10⁻¹ | 9.93 × 10⁻¹ |
| | gated; type V; alpha | | | | |
| Scn7a | sodium channel; voltage- | 9.10 × 10⁻¹ | −1.07 | 6.29 × 10⁻¹ | 8.78 × 10⁻¹ |
| | gated; type VII; alpha | | | | |
| Scn8a | sodium channel; voltage- | 5.76 × 10⁻¹ | 1.05 | 1.98 × 10⁻¹ | 5.85 × 10⁻¹ |
| | gated; type VIII; alpha | | | | |
| Scn9a | sodium channel; voltage- | 9.73 × 10⁻¹ | 1.29 | 2.62 × 10⁻⁴ | 1.21 × 10⁻² |
| | gated; type IX; alpha | | | | |
| Slc6a4 | solute carrier family 6 | 9.47 × 10⁻¹ | 4.38 | 1.56 × 10⁻⁵ | 1.55 × 10⁻³ |
| | (neurotransmitter | | | | |
| | transporter; serotonin); | | | | |
| | member 4 | | | | |
| Trak1 | trafficking protein; kinesin | 6.26 × 10⁻¹ | −1.01 | 7.24 × 10⁻¹ | 9.16 × 10⁻¹ |
| | binding 1 | | | | |
| Trpc6 | transient receptor potential | 9.10 × 10⁻¹ | 1.08 | 6.17 × 10⁻¹ | 8.73 × 10⁻¹ |
| | cation channel; subfamily C; | | | | |
| | member 6 | | | | |
| Trpm2 | transient receptor potential | 4.85 × 10⁻¹ | −1.02 | 6.78 × 10⁻¹ | 9.00 × 10⁻¹ |
| | cation channel; subfamily | | | | |
| | M; member 2 | | | | |
| Trpm3 | transient receptor potential | 6.66 × 10⁻¹ | 1.19 | 2.47 × 10⁻² | 2.19 × 10⁻¹ |
| | cation channel; subfamily | | | | |
| | M; member 3 | | | | |
| Trpm4 | transient receptor potential | 7.18 × 10⁻¹ | 1.05 | 6.82 × 10⁻¹ | 9.02 × 10⁻¹ |
| | cation channel; subfamily | | | | |
| | M; member 4 | | | | |
| Trpm7 | transient receptor potential | 5.27 × 10⁻¹ | 1.06 | 2.72 × 10⁻¹ | 6.59 × 10⁻¹ |
| | cation channel; subfamily | | | | |
| | M; member 7 | | | | |
| Trps1 | trichorhinophalangeal | 4.81 × 10⁻¹ | 1.03 | 6.17 × 10⁻¹ | 8.73 × 10⁻¹ |
| | syndrome I (human) | | | | |
| Trpv4 | transient receptor potential | 6.87 × 10⁻¹ | 1.21 | 7.37 × 10⁻¹ | 9.21 × 10⁻¹ |
| | cation channel; subfamily V; | | | | |
| | member 4 | | | | |
| Trpv6 | transient receptor potential | 9.40 × 10⁻¹ | −1.19 | 3.74 × 10⁻¹ | 7.42 × 10⁻¹ |
| | cation channel; subfamily V; | | | | |
| | member 6 | | | | |
| Unc80 | unc-80 homolog (C. | 6.84 × 10⁻¹ | 1.10 | 6.07 × 10⁻² | 3.44 × 10⁻¹ |
| | elegans) | | | | |

TABLE 7
Known nerve regeneration genes identified in our LMK235 treatment study and their mechanisms

| | | | LMK235 | |
|---|---|---|---|---|
| | | | Treated | |
| | | | FRICT-ION | LMK235 |
| | | | versus | Treated |
| | | FRICT-ION | Untreated | FRICT-ION |
| | | versus Naive | FRICT-ION | versus Naive |
| Reprogramming | Known Mechanisms and | Log2FC (p- | Log2FC (p- | Log2FC (p- |
| Gene | Pathways | value) | value) | value) |
| Oct3 | | | | |
| Oct4 (Pou5f1) | | | | |
| Sox2 | | 1.17 | 1.07 | 1.25 |
| | | (1.50 × 10⁻²) * | (2.68 × 10⁻¹) | (3.92 × 10⁻⁴) * |
| Klf4 | Obstructs DNA-binding and | −1.39 | 1.13 | −1.23 |
| | activation of phosphorylated Stat3 | (1 × 10⁻²) * | (3.69 × 10⁻¹) | (1.32 × 10⁻¹) |
| | in the JAK-STAT3 signaling | | | |
| | pathway | | | |
| c-Myc (Myc) | Dimerizes with Max (Myc- | 1.17 | −1.03 | 1.14 |
| | associated factor X) and then binds | (2.82 × 10⁻¹) | (8.43 × 10⁻¹) | (3.71 × 10⁻¹) |
| | to the E-box consensus sequence | | | |
| | in the promoter regions of genes to | | | |
| | regulate gene transcription for | | | |
| | many targets | | | |
| Nanog | | 1.05 | −1.03 | 1.01 |
| | | (7.87 × 10⁻¹) | (8.45 × 10⁻¹) | (9.35 × 10⁻¹) |
| Lin28b | Downregulates let-7 microRNAs | −1.20 | 1.07 | −1.13 |
| | to activate the Akt and mTOR | (2.93 × 10⁻²) * | (4.38 × 10⁻¹) | (1.50 × 10⁻¹) |
| | pathway whilst suppressing the | | | |
| | GSK3β pathway | | | |
| | Increases c-Myc transcription | | | |
| Brn2 | | | | |
| Ascl1 | | 1.10 | 1.05 | 1.16 |
| | | (5.31 × 10⁻¹) | (7.53 × 10⁻¹) | (3.45 × 10⁻¹) |
| Myt1l | | 1.19 | −1.17 | 1.03 |
| | | (1.97 × 10⁻⁶) * | (3.47 × 10⁻⁵) * | (5.06 × 10⁻¹) |
| NeuroD1 | | 1.04 | 1.21 | 1.26 |
| | | (8.26 × 10⁻¹) | (3.06 × 10⁻¹) | (2.15 × 10⁻¹) |
| NeuroG2 | | | | |
| Sox11 | Binds to DNA and activates or | 1.27 | −1.21 | 1.05 |
| | represses transcription of target | (4.44 × 10⁻³) * | (1.86 × 10⁻²) * | (5.84 × 10⁻¹) |
| | genes | | | |
| Klf1 | | | | |
| Klf2 | | −1.88 | 1.20 | −1.57 |
| | | (1.09 × 10⁻⁴) * | (2.62 × 10⁻¹) | (5.46 × 10⁻³) * |
| Klf5 | | 1.64 | −1.30 | 1.25 |
| | | (8.30 × 10⁻³) * | (1.26 × 10⁻¹) | (2.37 × 10⁻¹) |
| Klf6 | Functional interaction and co- | −1.02 | 1.05 | 1.03 |
| | expression/co-occupancy with | (7.34 × 10⁻¹) | (4.33 × 10⁻¹) | (6.62 × 10⁻¹) |
| | STAT3 in regulatory DNA in | | | |
| | growth-relevant gene networks | | | |
| Klf7 | Regulates gene expression | −1.03 | 1.15 | 1.12 |
| | | (8.32 × 10⁻¹) | (2.35 × 10⁻¹) | (3.37 × 10⁻¹) |
| Klf9 | Operates via Dual-specificity | 1.02 | −1.05 | −1.03 |
| | phosphatase 14 (Dusp14) to inhibit | (7.05 × 10⁻¹) | (2.24 × 10⁻¹) | (4.07 × 10⁻¹) |
| | MAPK signaling | | | |
| | Suppresses transcription of key | | | |
| | proteins in the cAMP pathway | | | |
| Klf13 | | 1.01 | −1.17 | −1.16 |
| | | (7.90 × 10⁻¹) | (1.37 × 10⁻³) * | (3.44 × 10⁻³) * |
| Klf14 | | | | |
| Klf15 | | −1.09 | −1.16 | −1.26 |
| | | (2.13 × 10⁻¹) | (2.77 × 10⁻²) * | (5.78 × 10⁻⁴) * |
| Klf16 | | 1.15 | −1.24 | −1.08 |
| | | (6.67 × 10⁻²) | (4.32 × 10⁻³) * | (3.35 × 10⁻¹) |
| Stat3 | Jak/Stat3 signaling pathway | 1.03 | −1.04 | −1.00 |
| | | (4.82 × 10⁻¹) | (4.34 × 10⁻¹) | (9.43 × 10⁻¹) |
| N-Myc (Mycn) | Dimerizes with Max (Myc- | 1.63 | 1.00 | 1.63 |
| | associated factor X) and then binds | (2.28 × 10⁻²) * | (9.95 × 10⁻¹) | (2.11 × 10⁻²) * |
| | to the E-box consensus sequence | | | |
| | in the promoter regions of genes to | | | |
| | regulate gene transcription for | | | |
| | many targets | | | |
| L-Myc (Mycl) | Dimerizes with Max (Myc- | 1.67 | −1.54 | 1.08 |
| | associated factor X) and then binds | (1.07 × 10⁻⁶) * | (2.16 × 10⁻⁵) * | (4.64 × 10⁻¹) |
| | to the E-box consensus sequence | | | |
| | in the promoter regions of genes to | | | |
| | regulate gene transcription for | | | |
| | many targets | | | |
| Sirt1 | Binds to the genomic DNA | 1.15 | 1.02 | 1.17 |
| | regions and inhibits miR-138 | (7.98 × 10⁻²) | (8.17 × 10⁻¹) | (4.59 × 10⁻²) * |
| | transcription | | | |
| LKB1 | Activates the kinases AMPKa | | | |
| | and NUAK1 with the ERK | | | |
| | signaling pathway while inhibiting | | | |
| | the kinase S6 and decreasing the | | | |
| | SAD-A and B kinase amounts | | | |
| p53 | c-Myc-TERT-p53 signaling | | | |
| | pathway | | | |
| TERT | c-Myc-TERT-p53 signaling | | | |
| | pathway | | | |

* indicates p-value < 0.05

### RT-PCR

Primers were ordered from Bio-Rad Laboratories, Inc. (Hercules, CA). Samples were run by UNM Human Tissue Repository Core facility. 400 μg of total RNA was transcribed into cDNA using iScript cDNA synthesis kit (Catalog #1708890, BioRad Laboratories, Inc., Hercules, CA, USA). Relative analysis of HDAC5 by Sybr Green qRT-PCR was run in order to validate the results observed via RNAseq. All primers, housekeeping gene primers, and the amplicon context sequence were purchased pre-validated from Bio-Rad Laboratories, Inc. (PrimePCR SYBR Green Assay: HDAC5, Mouse, qMmuCID0021326; Bio-Rad Laboratories, Inc., Hercules, CA). All qRT-PCR amplification reactions were done in 20 μl volumes in triplicate using iTaq Universal SYBR Green Supermix (Catalog #1725122, Bio-Rad Laboratories, Inc., Hercules, CA) in 96-well plates and run on a LightCycler 96 qRT-PCR system (Roche, Germany). The Amplification program used for amplification was an initial activation step of 95° C. for two minutes followed by 40 cycles at 95° C. for five seconds and 60° C. for 30 seconds, and concluding with a melt curve of 65° C.-95° C. with five seconds/step. qRT-PCR was done on the HDAC5 gene target and two housekeeping genes within each of the treated and untreated controls and experimental conditions. qRT-PCR data were analyzed using the 2-ΔΔcT method (Livak K J, Schmittgen T D, Methods 2001 December;25(4):402-408). Briefly, the mean ΔcT of each differentially expressed gene target assayed for, in both the experimental conditions and the untreated controls was calculated by subtracting the mean cT from the mean cT of the housekeeping genes values for each sample type. ΔΔcT was then calculated by subtracting the experimental ΔcT group from the control Act group. Expression fold change was expressed as 2-ΔΔcT.

### Microarray

For microarray analysis, TG were dissected, transferred to RNALATER (Life Technologies, Grand Island, NY), and stored at −80° C. Total RNA from ipsilateral TG was isolated using the RNeasy Mini Kit (Qiagen, Valencia, CA). Each replicate was derived from a single ipsilateral TG from n=6 mice/per group/per time point. RNA expression levels were determined with the Mouse Gene 2.0 Array (Affimetrix, Santa Clara, CA) at the University of Kentucky MicroArray Core Facility under the direction of Dr. Kuey-Chu Chen. Only numerical data is presented.

### Western Blot

Mice were euthanized with pentobarbital (FatalPlus, 480 mg/kg) and both ipsilateral and contralateral TG were removed. TG were washed immediately in PBS and stored at −80° C. Protein from TG tissue was extracted following homogenization using a pestle and 500 μL of 1× RIPA buffer (Thermo Fisher Scientific, Inc., Waltham, MA, Cat #89,900). Samples were put on a rocking shaker for two hours and then centrifuged, and the supernatant removed to a new tube. Sample was assayed for total protein (Bradford, Thermo Fisher Scientific, Inc., Waltham, MA). Samples were then prepared for electrophoresis by mixing with 2× sample buffer and boiling for five minutes at 100° C. for denaturation.

Proteins were loaded on a 12% Tris-Glycine polyacrylamide gradient gel (Bio-Rad Laboratories, Inc., Hercules, CA) and transferred to a PVDF membrane (MilliporeSigma, Burlington, MA). Membrane was blocked for an hour with 5% nonfat milk in TBST buffer at room temperature and incubated at 4° C. with anti-HDAC5 antibody overnight (Abcam, Cambridge, UK, #ab55403, #ab2772, #ab170935). The membrane was subsequently washed with TBST and then incubated with anti-rabbit secondary with HRP for one hour at room temperature (Abcam ab6721, diluted 1:1000 in TBST). The washing was repeated and then the blot developed with chemiluminescent substrate (Thermo Fisher Scientific, Inc., Waltham, MA Cat #32,106). The blot was then imaged using a Li-Cor Odyssey FC imaging system. Signal intensity was normalized to actin (Abcam ab8227, 1:2000) or tubulin (Abcam #ab10287), which was used as a loading control. Signal intensity was analyzed using ImageJ for comparisons.

### Cell Culture

Male mice were euthanized three weeks after inducing the FRICT-ION model. The TG were dissected, minced, and dissociated in an enzymatic combination containing sterile, calcium-free, and magnesium-free HBSS (Cat #14170112, Gibco), papain suspension (32.7 U/mgP, Cat #LS003126; Worthington, Lakewood, NJ), L-Cysteine (Cat #C7352-25g, Sigma), and saturated bicarbonate (Cat #S5761-500g, Sigma) solution. Secondary enzymatic combination solution to complete digestion contained HBSS, dispase II (2 mg/mL, cat #D4693-1g, Sigma), and collagenase type 2 (2 mg/mL, cat #LS004176; Worthington, Lakewood, NJ). After incubation for 20 minutes with gentle agitation under 37° C. for each enzymatic solution, TG neurons were triturated for 45 seconds in complete Leibovitz's L-15 medium (L-15 containing 5% fetal bovine serum, 1% antibacterial/antimycotic from 100 units/mL of penicillin, 100 μg/mL of streptomycin, 0.25 μg/mL of amphotericin B stock, and 2% of 1 M hepes solution, cat #11-415-064, Gibco). Then, the TG neurons were layered onto and centrifuged in a complete L-15 medium and PERCOLL gradient (GE Healthcare Bio-Sciences AB, Uppsala, Sweden; density=1.130 g/mL, 12.5% PERCOLL layered over 28% PERCOLL, cat #89428-522, VWR) for 10 minutes at 1300×g and again in complete L-15 medium for six minutes at 1000×g. After cell pellet formed, supernatant was carefully aspirated, and pellet was resuspended in DMEM medium (10% fetal bovine serum and 1% antibacterial/antimycotic). Primary cultures were established using 5% CO2 with DMEM culture medium supplemented with 10% fetal bovine serum, 1% antibiotic-antimycotic solution (Sigma). Cells were plated at a density of 1,270 cells/mm² (79% viability) on 12 mm² poly-d-lysine-coated glass coverslips. Electrophysiological recordings were performed 18-40 hours after plating. LMK235 (13 μM) or vehicle control (0.1% DMSO) was applied to culture media for one hour prior to recording.

### Power, Statistical and Data Analysis

The power analysis provided group size information to achieve statistical significance in all study groups based on the mean reaction time, staining intensity, or response increase among groups. Behaviors were compared among groups to their baselines (week 0), sham controls, untreated nerve injured mice, and mice with pre-HDACi treatment or post-HDACi treatment as the apriori contrasts of interest. Studies with n=6 in each group, included naïve, sham, and nerve injury animals, were repeated at least twice to accumulate cohorts of 12 animals to accommodate the behavioral, fixed, and fresh tissue studies. Behavioral test results are reported using non-parametric comparisons. Statistical analysis was performed in PRISM 8.0 software (GraphPad Software, Inc., San Diego, CA). Appropriate statistical tests and post hoc tests are specified in the corresponding data figures. In all cases an α type-I error value of 0.05 was accepted for significant differences.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of treating neuropathic pain in a subject, the method comprising administering to the subject an amount of LMK235 effective to alleviate neuropathic pain experienced by the subject.

2. The method of claim 1, wherein the neuropathic pain is chronic.

3. The method of claim 1, wherein the effective amount of LMK235 is an amount effective to reduce mechanical sensitivity in the subject compared to a vehicle-treated control.

4. The method of claim 1, wherein the effective amount of LMK235 is an amount effective to reduce cold sensitivity in the subject compared to a vehicle-treated control.

5. The method of claim 1, wherein the effective amount of LMK235 is an amount effective to reduce anxiety-related behaviors associated with experiencing neuropathic pain compared to a vehicle-treated control.

6. The method of claim 1, wherein the effective amount of LMK235 is an amount effective to reduce excitability of isolated trigeminal ganglia neurons from an injured nerve injured compared to a vehicle-treated control.

7. The method of claim 6, wherein the effective amount of LMK235 is an amount effective to reverse one or more epigenetic change that resulted from injury to the injured nerve.

8. The method of claim 1, wherein the effective amount of LMK235 is an amount to reduce sag ratio in trigeminal neurons compared to injured trigeminal neurons untreated with LMK235.

9. The method of claim 1, wherein the effective amount of LMK235 is an amount from 5 ng/kg to 50 mg/kg.

10. The method of claim 9, wherein the effective amount of LMK235 is from 100 ng/kg to 100 g/kg.

11. The method of claim 10, wherein the effective amount of LMK235 is from 1 μg/kg to 5 μg/kg.

12. A method of suppressing histone deacetylase 5 (HDAC5) expression in a cell, the method comprising contacting the cell with an amount of LMK235 effective to suppress expression of HDAC5.

13. A method of suppressing histone deacetylase 5 (HDAC5) expression in a subject, the method comprising administering LMK235 to the subject in an amount of LMK235 effective to suppress expression of HDAC5 in one or more tissues of the subject.

14. The method of claim 13, wherein the subject has, or is at risk of having, a condition caused at least in part by overexpression of HDAC5.

15. The method of claim 14, wherein the condition is diabetic neuropathy, myelofibrosis, periodontitis, osteoporosis, osteopetrosis, bacterial-induced osteolysis, nerve injury, brain injury, systemic sclerosis, rheumatoid arthritis, diabetic kidney disease, acute kidney injury, polycystic kidney disease, polycystic ovary, or an inflammatory gastrointestinal syndrome.

16. The method of claim 15, wherein the inflammatory gastrointestinal syndrome comprises Crohn's disease, inflammatory bowel syndrome, or intestinal sepsis.

17. The method of claim 14, wherein the condition is a cancer.

18. The method of claim 17, wherein the cancer comprises breast cancer, lung cancer, esophagus cancer, colon cancer, prostate cancer, ovarian cancer, hepatocellular carcinoma, colorectal cancer, neuroblastoma, or head and neck cancer.

19. The method of claim 17, wherein the cancer comprises glioblastoma, lymphoma, or multiple myeloma.

20. The method of claim 17, wherein LMK235 at least partially reverses resistance to tamoxifen, aromatase inhibitor therapy, or both.

21. The method of claim 13, wherein administering LMK235 to the subject improves cardiac dysfunction, pathological ventricular remodeling, or both.
